(19) United States
(12) Patent Application Publication
(10) Pub. No.: US 2026/0233203 A1
(43) Pub. Date: Aug. 13, 2026

(54) Catalyst Compositions and Processes for Making and Using Same

(71) Applicant: ExxonMobil Technology and Engineering Company, Spring, TX (US)

(72) Inventors: Yi Du, Coopersburg, PA (US); Bradley D. Wooler, Allentown, PA (US); Xiaoying Bao, Houston, TX (US)

(21) Appl. No.: 19/154,940

(22) PCT Filed: Jan. 25, 2024
(86) PCT No.: PCT/US2024/012861

§ 371 Date: Aug. 8, 2025

Related U.S. Application Data
(60) Provisional application No. 63/485,950, filed on Feb. 20, 2023.

(51) Int. Cl. B01J 23/62; B01J 23/02; B01J 35/30; B01J 35/61; B01J 37/00; B01J 37/02; B01J 37/03; B01J 37/04; B01J 37/06; B01J 37/08; C07C 5/333

(52) U.S. Cl. CPC B01J 23/626; B01J 23/02; B01J 35/394; B01J 35/615; B01J 37/009; B01J 37/0207; B01J 37/0236; B01J 37/036; B01J 37/04; B01J 37/06; B01J 37/08; C07C 5/3337; C07C 2523/14; C07C 2523/42; C07C 2523/62

(57) ABSTRACT

Catalyst compositions and processes for making and using same. The catalyst composition can include up to 6 wt % of Pt and up to 10 wt % of a promoter comprising Sn, Cu, Au, Ag, Ga, a combination thereof, or a mixture thereof disposed on a support, based on the weight of the support. The support can include a mixed Mg/A metal oxide. The support can have a surface area>300 m²/g.

### CROSS-REFERENCE TO RELATED APPLICATION

This application claims priority to and the benefit of U.S. Provisional Application No. 63/485,950 having a filing date of Feb. 20, 2023, the disclosure of which is incorporated herein by reference in its entirety.

### FIELD

This disclosure relates to catalyst compositions and processes for making and using same.

### BACKGROUND

Catalytic reforming or dehydrogenation, dehydroaromatization, and/or dehydrocyclization of alkane and/or alkyl aromatic hydrocarbons are industrially important chemical conversion processes that are endothermic and equilibrium-limited. The reforming or dehydrogenation, dehydroaromatization, and/or dehydrocyclization of alkanes, e.g., C₁-C₁₂ alkanes, and/or alkyl aromatics, e.g., ethylbenzene, can be done through a variety of different catalyst compositions such as the Pt-based, Ni-based, Pd-based, Ru-based, Re-based, Cr-based, Ga-based, V-based, Zr-based, In-based, W-based, Mo-based, in-based, and Fe-based systems.

Precious metals used in synthesizing a catalyst composition can be expensive and can contribute significantly to the capital operating expenses and/or operating expenses of a chemical plant. During the synthesis of the catalyst composition, it is believed that the distribution of the precious metals about the support is an important aspect in formulating an active catalyst. The currently known synthesis processes that are used to produce catalyst compositions yield catalyst compositions that exhibit less activity than desired.

There is a need, therefore, for improved catalyst compositions and processes for making and using same. This disclosure satisfies this and other needs.

### SUMMARY

Catalyst compositions and processes for making and using same are provided. In some embodiments, the catalyst composition can include up to 6 wt % of Pt and up to 10 wt % of a promoter comprising Sn, Cu, Au, Ag, Ga, a combination thereof, or a mixture thereof disposed on a support, where all weight percent values are based on the weight of the support. The support can include a mixed Mg/Al metal oxide. The support can have a surface area >300 m²/g.

In some embodiments, the process for making a catalyst composition can include (I) combining a first compound that includes Mg, a second compound that includes Al, a first base compound, water, and optionally a Pt-containing compound and/or a promoter-containing compound under agitation conditions for a time sufficient to produce a slurry or a gel comprising a plurality of precursor support particles that include water disposed thereon and/or therein. The process can also include (II) contacting the slurry or the gel with an organic solvent to displace at least a portion of the water from the slurry or the gel to produce a plurality of precursor support particles that can include the organic solvent disposed thereon and/or therein, where optionally a Pt-containing compound and/or a promoter-containing compound can be contacted with (i) the slurry or the gel or (ii) with the plurality of precursor support particles that can include the organic solvent disposed thereon and/or therein. The process can also include (III) removing at least a portion of the organic solvent from the plurality of precursor support particles to produce a plurality of dried precursor support particles, where optionally a Pt-containing compound and/or a promoter-containing compound can be contacted with the dried precursor support particles. The process can also include (IV) heating the plurality of dried precursor support particles to a temperature of at least 350° C., to produce a plurality of calcined support particles, where optionally a Pt-containing compound and/or a promoter-containing compound can be contacted with the plurality of calcined support particles. The Pt-containing compound and the promoter-containing compound can independently be present in at least one of steps (I), (II). (III), and (IV). The catalyst composition can include up to 6 wt % of Pt and up to 10 wt % of the promoter, based on the weight of the plurality of calcined support particles. The promoter can include Sn, Cu, Au. Ag, Ga, a combination thereof, or a mixture thereof. The plurality of calcined support particles can include a mixed Mg/Al metal oxide. The plurality of calcined support particles can have a surface area ≥300 m²/g.

In some embodiments, a process for upgrading a hydrocarbon can include (I) contacting a hydrocarbon-containing feed with a catalyst composition that can include Pt and a promoter disposed on a support to effect one or more of dehydrogenation, dehydroaromatization, and dehydrocyclization of at least a portion of the hydrocarbon-containing feed to produce a coked catalyst composition and an effluent that can include one or more upgraded hydrocarbons and molecular hydrogen. The hydrocarbon-containing feed can include one or more of C₂-C₁₆ linear or branched alkanes, or one or more of C₄-C₁₆ cyclic alkanes, or one or more C₈-C₁₆ alkyl aromatics, or a mixture thereof. The catalyst composition can include up to 6 wt % of Pt and up to 10 wt % of the promoter, based on the weight of the support. The promoter can include Sn, Cu, Au, Ag, Ga, a combination thereof, or a mixture thereof. The support can include a mixed Mg/Al metal oxide. The support can have a surface area ≥300 m²/g.

### DETAILED DESCRIPTION

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other steps, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for acquiring the measurement.

Certain embodiments and features are described herein using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated.

The indefinite article “a” or “an”, as used herein, means “at least one” unless specified to the contrary or the context clearly indicates otherwise. Thus, embodiments using “a reactor” or “a conversion zone” include embodiments where one, two or more reactors or conversion zones are used, unless specified to the contrary or the context clearly indicates that only one reactor or conversion zone is used.

The term “hydrocarbon” means (i) any compound consisting of hydrogen and carbon atoms or (ii) any mixture of two or more such compounds in (i). The term “Cn hydrocarbon.” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of these compounds at any proportion. A “Cm to Cn hydrocarbon” or “Cm-Cn hydrocarbon,” where m and n are positive integers and m<n, means any of Cm, Cm+1. Cm+2, . . . , Cn-1, Cn hydrocarbons, or any mixtures of two or more thereof. Thus, a “C₂ to C₃ hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn-hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

For the purposes of this disclosure, the nomenclature of elements is pursuant to the version of the Periodic Table of Elements (under the new notation) as provided in Hawley's Condensed Chemical Dictionary, 16^{th} Ed., John Wiley & Sons, Inc., (2016). Appendix V. For example, a Group 2 element includes Mg, a Group 8 element includes Fe, a Group 9 element includes Co, a Group 10 element includes Ni, and a Group 13 element includes Al. The term “metalloid”, as used herein, refers to the following elements: B, Si, Ge, As, Sb, Te, and At. In this disclosure, when a given element is indicated as present, it can be present in the elemental state or as any chemical compound thereof, unless it is specified otherwise or clearly indicated otherwise by the context.

The term “alkane” means a saturated hydrocarbon. The term “cyclic alkane” means a saturated hydrocarbon comprising a cyclic carbon ring in the molecular structure thereof. An alkane can be linear, branched, or cyclic.

the term “aromatic” is to be understood in accordance with its art-recognized scope, which includes alkyl substituted and unsubstituted mono- and polynuclear compounds.

The term “rich” when used in phrases such as “X-rich” or “rich in X” means, with respect to an outgoing stream obtained from a device, e.g., a conversion zone, that the stream comprises material X at a concentration higher than in the feed material fed to the same device from which the stream is derived. The term “lean” when used in phrases such as “X-lean” or “lean in X” means, with respect to an outgoing stream obtained from a device, e.g., a conversion zone, that the stream comprises material X at a concentration lower than in the feed material fed to the same device from which the stream is derived.

The term “mixed metal oxide” refers to a composition that includes oxygen atoms and at least two different metal atoms that are mixed on an atomic scale. For example, a “mixed Mg/Al metal oxide” has O, Mg, and Al atoms mixed on an atomic scale and is substantially the same as or identical to a composition obtained by calcining an Mg/Al hydrotalcite that has the general chemical formula

$$[ {Mg}_{( 1 - x )} {{Al}_{x} ( OH )}_{2} ( {A}_{\frac{x}{n}}^{n -} ) \cdot {mH}_{2} O ] ,$$

where A is a counter anion of a negative charge n. A material consisting of nm sized MgO particles and nm sized Al₂O₃ particles mixed together is not a mixed metal oxide because the Mg and Al atoms are not mixed on an atomic scale but are instead mixed on a nm scale.

The terms “calcination” and “calcining” refer to heating a material, e.g., a synthesized catalyst or a support, to a temperature of 350° C., or more under any atmosphere, e.g., an oxidizing atmosphere, an inert atmosphere, or a reducing atmosphere. The term “calcined” refers to a material, e.g., a synthesized catalyst or a support, that has been subjected to calcination/calcining.

The term “selectivity” refers to the production (on a carbon mole basis) of a specified compound in a catalytic reaction. As an example, the phrase “an alkane hydrocarbon conversion reaction has a 100% selectivity for an olefin hydrocarbon” means that 100% of the alkane hydrocarbon (carbon mole basis) that is converted in the reaction is converted to the olefin hydrocarbon. When used in connection with a specified reactant, the term “conversion” means the amount of the reactant consumed in the reaction. For example, when the specified reactant is propane, 100% conversion means 100% of the propane is consumed in the reaction. In another example, when the specified reactant is propane, if one mole of propane converts to one mole of methane and one mole of ethylene, the selectivity to methane is 33.3% and the selectivity to ethylene is 66.7%. Yield (carbon mole basis) is conversion times selectivity.

In this disclosure, “A, B, . . . , or a combination thereof” means “A, B, . . . , or any combination of any two or more of A, B, . . . ” “A, B, . . . , or a mixture thereof” means “A, B, . . . , or any mixture of any two or more of A, B, . . . ”

### Catalyst Composition

In some embodiments, the catalyst composition can include up to up to 6 wt % of Pt and up to 10 wt % of a promoter that can include Sn, Cu, Au, Ag, Ga, a combination thereof, or a mixture thereof disposed on a support, w here all weight percent values are based on the weight of the support. The catalyst composition can include 0.001 wt %, 0.002 wt %, 0.003 wt %, 0.004 w t %, 0.005 wt %, 0.006 wt %, 0.007 wt %, 0.008 wt %, 0.009 wt %, 0.01 wt %, 0.015 wt %, 0.02 wt %, 0.025 wt %, 0.03 wt %, 0.05 wt %, 0.07 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 1.3 wt %, 1.5 wt %, 1.7 wt %, 2 wt %, 2.3 wt %, 2.5 wt %, 2.7 wt %, 3 wt %, 3.3 wt %, 3.5 wt %, 3.7 wt %, 4 wt %, 4.3 wt %, 4.5 wt %, 4.7 wt/6, 5 wt %, 5.3 wt %, 5.5 wt %, 5.7 wt %, or 6 wt % of Pt disposed on the support, based on the weight of the support.

The catalyst composition can include the promoter in an amount of 0.01 wt %, 0.1 wt %, 0.2 wt %, 0.25 wt %, 0.3 wt %, 0.4 wt %, 0.5 wt %, 0.6 wt %, 0.7 wt %, 0.8 wt %, 0.9 wt %, or 1 wt % to 2 wt %, 3 wt %, 4 wt %, 5 wt %, 6 wt %, 7 wt %, 8 wt %, 9 wt %, or 10 wt % disposed on the support, based on the weight of the support. In some embodiments, the promoter can be or can include, but is not limited to, Sn, Cu, Au. Ag, Ga, a combination thereof, or a mixture thereof. In some embodiments, the promoter can be associated with the Pt. For example, the promoter and the Pt disposed on the support can form Pt-promoter clusters that can be dispersed on the support. The promoter can improve the selectivity/activity/longevity of the catalyst composition for a given upgraded hydrocarbon. In some embodiments, the promoter can improve the propylene selectivity of the catalyst composition when the hydrocarbon-containing feed includes propane.

In some embodiments, the catalyst composition can optionally include one or more alkali metal elements in an amount of up to 5 wt % disposed on the support, based on the weight of the support. The catalyst composition can include 0.01 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 2 wt %, 3 wt %, 1.4 wt %, or 5 wt % of the alkali metal element disposed on the support, based on the weight of the support. The alkali metal element, if present, can be or can include, but is not limited to, Li, Na, K, Rb, Cs, or a combination thereof, or a mixture thereof. In at least some embodiments, the alkali metal element ca be or can include K and/or Cs. In some embodiments, the alkali metal element, if present, can improve the selectivity of the catalyst composition for a given upgraded hydrocarbon.

The support can be or can include, but is not limited to, a mixed Mg/Al metal oxide. The support can include ≥0.5 wt %, ≥1 wt %, ≥2 wt %, ≥3 wt5, ≥4 wt %, 5 wt %, ≥10 wt %, or ≥20 wt %, ≥40 wt %. % 80 wt %, or ≥90 w t % of Mg, based on the weight of the support. In some embodiments, the support can include Mg in a range of from 0.5 wt %, 3 wt %, 5 wt %, or 10 wt % to 30 wt %, 50 wt %, 70 wt %, or 90 wt %, based on the weight of the support.

In some embodiments, the support can be or can include a mixed Mg/Al metal oxide having the same or similar structure as a compound produced or obtained by calcining hydrotalcite but made via an alternative process such that the support has a greater surface area than the compound produced or obtained by calcining hydrotalcite. For example, in some embodiments, the support can be or can include a mixed Mg/Al metal oxide produced by combining a first compound that includes Mg and a second compound that includes Al and additional ingredients to produce a slurry or a gel that can be processed according to one or more synthesis procedures described in more detail below to produce the mixed Mg/Al metal oxide. In some embodiments, the mixed Mg/Al metal oxide making up the support or at least a portion of the support, the mixed Mg/Al metal oxide can have a weight ratio of Mg to Al in a range of from 0.001, 0.005, 0.01, 0.05, 0.1, 0.5, 1, 1.5, 2, 2.5, 3, 3.5, 4, 4.5, or 5 to 6, 10, 125, 25, 50, 75, 100, 150, 200, 250, 300, 350, 400, 450, 500, 550, 600, 650, 700, 750, 800, 850, 900, 950, or 1,000.

In some embodiments, the support can have a surface area ≥125 m²/g, ≥150 m²/g, ≥175 m²/g, ≥200 m²/g, ≥225 m²/g, ≥250 m²/g, ≥275 m²/g, ≥300 m²/g, ≥303 m²/g, 305 m²/g, ≥310 m²/g, ≥315 m²/g, ≥320 m²/g, 325 m²/g, 330 m²/g, ≥335 m²/g, ≥340 m²/g, ≥345 m²/g, 350 m²/g, ≥355 m²/g, 360 m²/g, ≥365 m²/g, ≥370 m²/g, ≥375 m²/g, ≥380 m²/g, ≥385 m²/g, ≥390 m²/g, 395 m²/g, ≥400 m²/g, ≥425 m²/g, ≥450 m²/g, ≥475 m²/g, or ≥500 m²/g. In some embodiments, the support can have a surface area in a range of from ≥200 m²/g, ≥250 m²/g, ≥275 m²/g, ≥300 m²/g, ≥325 m²/g, ≥350 m²/g, or ≥370 m²/g to ≤400 m²/g, ≤425 m²/g, ≤450 m²/g, or ≤475 m²/g, ≤500 m²/g, 525 m²/g, 550 m²/g, ≤575 m²/g, ≤600 m²/g, ≤625 m²/g, ≤650 m²/g. The surface area of the catalyst particles can be measured according to the Brunauer-Emmett-Teller (BET) method using adsorption-desorption of nitrogen (temperature of liquid nitrogen, 77 K) with a Micromeritics 3flex instrument after degassing of the powders for 4 hrs at 350° C. More information regarding the method can be found, for example, in “Characterization of Porous Solids and Powders: Surface Area, Pore Size and Density,” S. Lowell et al., Springer, 2004.

In some embodiments, when the Pt and the promoter are added to the support during synthesis of the catalyst composition, the support can have a surface area ≥300 m²/g, ≥300 m²/g, ≥303 m²/g, 305 m²/g, ≥310 m²/g, ≥315 m²/g, 320 m²/g, ≥325 m²/g, ≥330 m²/g, p 335 m²/g, ≥340 m²/g, ≥345 m²/g, ≥350 m²/g, ≥355 m²/g, 360 m²/g, ≥365 m²/g, ≥370 m²/g, ≥375 m²/g, % 380 m²/g, ≥385 m²/g, ≥390 m²/g, ≥395 m²/g, ≥400 m²/(, ≥425 m²/g, 450 m²/g, ≥475 m²/g, or ≥500 m²/g. In some embodiments, as further described below, the Pt and promoter can be added to the support during synthesis of the catalyst composition in the form of a Pt-containing compound and a promoter-containing compound.

In some embodiments, a molar ratio of the Mg to the Pt present in the catalyst composition can be in a range from 0.24, 0.5, 1, 10, 50, 100, 300, 450, 600, 800, 1,000, 1.200, 1,500, 1.700, or 2.000 to 3.000, 3,500, 4,000, 4,500, 5,000, 5,500, 6,000, 6,500, 7,000, 7.500, 8.000, 8,500, 9.000, 9,500, 10.000, 15,000, 20.000, 25.000, 30.000, 35,000, 40,000, 45.000, 50.000, 55.000, 60,000, 65,000, 70,000, 75,000, 80.000, 85.000, 90.000, 95.000, 100,000, 200,000, 300.000, 400,000, 500,000, 600,000, 700,000, 800000, or 900,000.

In some embodiments, the catalyst composition can be in the form of catalyst particles or a monolithic structure. If the catalyst composition is in the form of catalyst particles, the catalyst particles can have a median particle size in a range of from 1 μm, 5 μm, 10 μm, 20 μm, 40 μm, or 60 μm to 80 μm, 100 μm, 115 μm, 130 μm, 150 μm, 200 μm, 300 μm or 400, or 500 μm. The catalyst particles can have an apparent loose bulk density in a range from 0.3 g/cm³, 0.4 g/cm³, 0.5 g/cm³, 0.6 g/cm³, 0.7 g/cm³, 0.8 g/cm³, 0.9 g/cm³, or 1 g/cm³ to 1.1 g/cm³, 1.2 g/cm³, 1.3 g/cm³, 1.4 g/cm³, 1.5 g/cm³, 1.6 g/cm³, 1.7 g/cm³, 1.8 g/cm³, 1.9 g/cm³, or 2 g/cm³, as measured according to ASTM D7481-18 modified with a 10, 25, or 50 mL graduated cylinder instead of a 100 or 250 mL graduated cylinder. In some embodiments, the catalyst particles can have an attrition loss after one hour of 5 wt %, ≤4 wt %, 3 wt %, ≤2 wt %, ≤1 wt %, ≤0.7 wt %, ≤0.5 wt %, ≤0.4 wt %, ≤0.3 wt %, ≤0.2 wt %, ≤0.1 wt %, ≤0.07 wt %, or ≤0.05 wt %, as measured according to ASTM D5757-11(2017). The morphology of the particles can be largely spherical so that they are suitable to run in a fluid bed reactor. In some embodiments, the catalyst particles can have a size and density that is consistent with a Geldart A or Geldart B definition of a fluidizable solid.

### Processes for Making the Catalyst Composition

The process for making the catalyst composition can include preparing a slurry or gel that can include, but is not limited to, combining a Mg-containing compound, an Al-containing compound, a first base compound, water, and optionally a Pt-containing compound and/or optionally a promoter-containing compound and/or optionally an alkali metal element-containing compound that includes at least one of Li, Na, K, Rb, and Cs to produce a mixture. The mixture can be subjected to agitation conditions for a time sufficient to produce the slurry or a gel that can include a plurality of precursor support particles that can include water disposed thereon and/or therein. The Mg-containing compound, the Al-containing compound, the first base compound, water, and optionally the Pt-containing compound optionally a promoter-containing compound and/or optionally an alkali metal element-containing compound can be combined with one another within any suitable vessel, which can be an open vessel or a closed vessel with respect to the atmosphere. In some embodiments, the agitation conditions can be imparted to the mixture via milling, mixing, blending, stirring, ultrasonic sound waves, or any other suitable processes capable of agitating the mixture.

In some embodiments, the mixture can be agitated for a time period in a range from 1 hour, 2 hours, 3 hours, 4 hours, 5 hours, 7 hours, 10 hours, 13 hours, or 15 hours to 17 hours, 19 hours, or 21 hours, 23 hours, 26 hours, 29 hours, 33 hours, 36 hours, 40 hours or more to produce the slurry or gel. In some embodiments, the mixture can be agitated at a temperature in a range from 10° C., 20° C., 30° C., 40° C., 50° C., 60° C., or 70° C., to 80° C., 90° C., 100° C., 110° C., 120° C., 130° C., 140° C., or 150° C., to produce the slurry or gel. In some embodiments, during agitation of the mixture, the mixture can be at an ambient temperature. In some embodiments, the mixture can be agitated under a vacuum, an ambient pressure, or at an elevated pressure relative to ambient pressure.

In some embodiments, the Mg-containing compound, the Al-containing compound, the first base compound, water, and optionally the Pt-containing compound and/or optionally a promoter-containing compound and/or optionally an alkali metal element-containing compound can be combined with one another simultaneously or in any sequences with respect to one another to produce the mixture. In some embodiments, a first portion of the water, the Mg-containing compound, and the Al-containing compound can be combined with one another to produce a first aqueous mixture and a second portion of the water and the first base compound can be combined with one another to produce a second aqueous mixture. In such embodiment, the optional Pt-containing compound and/or the optional promoter, and/or the optional alkali metal element-containing compound if present, can also independently be combined with the first portion of the water and/or the second portion of the water to produce the first aqueous mixture and/or the second aqueous mixture, respectively. In some embodiments, the amount of the first base compound in the second aqueous mixture can be sufficient to provide the second aqueous mixture with a pH in a range of from 7, 7.5, 8, 8.5, or 9 to 10, 10.5, 11, 11.5, or 12.

The first and second aqueous mixtures can be combined with one another to produce the mixture that includes the Mg-containing compound, the Al-containing compound, the first base compound, water, and optionally the Pt-containing compound and/or optionally the promoter-containing compound and/or optionally the alkali metal element-containing compound. In some embodiments, the first and second aqueous mixtures can be combined to produce the mixture and then agitation can begin. In other embodiments, the first and second aqueous mixtures can be combined under the agitation conditions to produce the mixture. In some embodiments, the first aqueous mixture can be added to the second aqueous mixture, the second aqueous mixture can be added to the first aqueous mixture, or the first and second aqueous mixtures can simultaneously be introduced into the vessel to produce the mixture. In some embodiments, a second base compound can be combined to maintain a pH of the mixture in a range of from 7, 7.5.8, 8.5, or 9 to 10, 10.5, 11, 11.5, or 12 as the first and second aqueous mixtures are combined with one another and subjected to the agitation conditions.

The mixture that includes the Mg-containing compound, the Al-containing compound, the first base compound, water, and optionally the Pt-containing compound and/or optionally a promoter-containing compound and/or optionally an alkali metal element-containing compound that includes at least one of Li, Na, K, Rb, and Cs can be in the form of an aqueous solution. Similarly, when the first and second aqueous mixtures are produced the first and second aqueous mixtures can be in the form of an aqueous solution and, when combined with one another, the mixture can also be in the form of an aqueous solution.

In some embodiments, the Mg-containing compound can be in the form of a nitrate, a hydrated nitrate, an oxide, a hydroxide, a hydrated carbonate, a salt, a clay containing Mg, a phosphate, a halide, a halate, a sulfate, a sulfide, a borate, an aluminate, an aluminosilicate, a silicate, a carbonate, a hydrated carbonate, metaphosphate, a tungstate, a molybdate, or a mixture thereof. In some embodiments, the Mg-containing compound can be in the form of a hydrated magnesium nitrate having a formula of Mg(NO₃)₂(H₂O)ₓ, where x=6, 2, or 0; a magnesium oxide; a magnesium hydroxide, hydromagnesite (a hydrated magnesium carbonate mineral, Mg₅(CO₃)₄(OH)₂·4H₂O); a magnesium salt; a magnesium-containing clay; or a mixture thereof.

In some embodiments, the Al-containing compound can be in the form of a nitrate, a hydrated nitrate, an oxide, a carbonate, a hydrated carbonate, a sodium aluminum carbonate mineral, a hydrated aluminum carbonate mineral, a halide, or a mixture thereof. In some embodiments, the Al-containing compound can be or can include, but is not limited to, a hydrated aluminum nitrate having a formula of Al(NO₃)₃(H₂O)_{y}, where y=9 or 0; aluminum oxide; (NaACO₃(OH)₂; (Al₅(CO₃)(OH)₁₃·5(H₂O)); (A₁₄(CO₃)₃(OH)₃₆·nH₂O); AlC₃, or a mixture thereof.

In some embodiments, the first base compound can be or can include, but is not limited to, sodium carbonate, sodium nitrate, sodium chloride, sodium sulfate, sodium hydroxide, ammonium hydroxide, potassium hydroxide, urea, hexamethyl tetraamines, or a mixture thereof. In some embodiments, the second base compound can be or can include, but is not limited to, sodium carbonate, sodium nitrate, sodium chloride, sodium sulfate, sodium hydroxide, ammonium hydroxide, potassium hydroxide, urea, hexamethyl tetraamines, or a mixture thereof. In some embodiments, the first base compound and the second base compound can be the same or different with respect to one another. In some embodiments, the water can be or can include, but is not limited to, tap water, distilled water, and/or deionized water.

In some embodiments, the Pt-containing compound can be or can include, but is not limited to, chloroplatinic acid hexahydrate, tetraammineplatmnum(II) nitrate, platinum(II) acetylacetonate, platinum(II) bromide, platinum(II) iodide, platinum(II) chloride, platinum(IV) chloride, platinum(II)diammine dichloride, ammonium tetrachloroplatinate(II), tetraammineplatinum(il) chloride hydrate, tetraammineplatinum(II) hydroxide hydrate, platinum (II) oxalate, or any mixture thereof.

In some embodiments, the promoter-containing compound can be or can include, but is not limited to, tin(IV) chloride pentahydrate, tin(II) chloride dihydrate, tin(II) bromide, tin(IV) bromide, tin(II) acetylacetonate, tin(II) acetate, tin(IV) acetate, tin(II) oxalate, tin(IV) oxalate, silver(I) nitrate, gold(III) nitrate, copper(II) nitrate, gallium(III) nitrate, or any mixture thereof.

In some embodiments, the alkali metal element-containing compound can be or can include, but is not limited to, lithium nitrate, sodium nitrate, potassium nitrate, rubidium nitrate, cesium nitrate, or any mixture thereof.

The process can also include contacting the slurry or gel with an organic solvent to displace at least a portion of the water from the slurry or the gel to produce a plurality of precursor support particles that include the organic solvent disposed thereon and/or therein. In some embodiments, the slurry or gel can be contacted with the optional Pt-containing compound and/or the optional promoter containing compound and/or the optional alkali metal element-containing compound before, simultaneously with, and/or after the slurry or gel is contacted with the organic solvent. In some embodiments, the plurality of precursor support particles that include the organic solvent disposed thereon and/or therein can be contacted with the optional Pt-containing compound and/or the optional promoter containing compound and/or the optional alkali metal element-containing compound. In some embodiments, the organic solvent can be or can include, but is not limited to, ethanol, propanol, butanol, pentanol, acetone, ethyl acetate, or a mixture thereof.

An excess of the organic solvent can be separated from the plurality of precursor support particles via any suitable apparats or process. In some embodiments, an excess of the organic solvent can be separated from the plurality of precursor support particles via filtration, centrifugation, or a combination thereof.

In some embodiments, contacting the slurry or gel with the organic solvent can be carried out in a single contact step or two or more contact steps. For example, in some embodiments, the slurry or gel can be filtered and washed with a first quantity of the organic solvent to produce a filtered product that can be free of or contain a reduced amount of water disposed thereon and/or therein that can include the organic solvent disposed thereon and/or therein. In another example, in other embodiments, the slurry or gel can be filtered and washed with water followed by a first quantity of the organic solvent to produce a first filtered product. The first filtered product, without drying, can be combined with a second quantity of the organic solvent under agitation conditions for a for a time period in a range from 15 minutes, 30 minutes, 1 hour, 3 hours, or 5 hours to 7 hours, 10 hours, 12 hours, 15 hours, 20 hours, or more to produce the plurality of precursor support particles that include the organic solvent disposed thereon and/or therein. In some embodiments, excess organic solvent can be separated, e.g., via filtration, from the plurality of precursor support particles.

When two or more contact steps are used, a composition of the first quantity of the organic solvent and a composition of the second quantity of the organic solvent can be the same or different with respect to one another. The first and second quantities of organic solvent can independently be or include, ethanol, propanol, butanol, pentanol, acetone, ethyl acetate, or a mixture thereof. In some embodiments, a weight ratio of the second quantity of the organic solvent to the first filtered product can be in a range from 0.1:1, 0.5:1, or 1:1 to 5:1, 10:1, 20:1, or 30:1.

The process can also include removing at least a portion of the organic solvent from the plurality of precursor support particles to produce a plurality of dried precursor support particles. In some embodiments, the process can optionally include contacting the plurality of dried precursor with a Pt-containing compound and/or a promoter-containing compound and/or an alkali metal element-containing compound.

In some embodiments, at least a portion of the organic solvent can be removed from the plurality of precursor support particles by drying the plurality of precursor support particles (i) under an atmosphere that can be or can include, but is not limited to, nitrogen, argon, carbon dioxide, methane, air, or any mixture thereof or (ii) under a vacuum in the substantial absence of an atmosphere to produce the plurality of dried precursor support particles. In such embodiment, the plurality of precursor support particles can be at a temperature in a range from 20° C., 50° C., 100° C., or 150° C., to 200° C., 250° C., 300° C., or 340° C. In such embodiment, the plurality of precursor support particles can be dried for a time period in a range from 30 minutes, 1 hour, 1.5 hours, 2 hours, 5 hours, 7 hours, or 10 hours to 15 hours, 17 hours, 20 hours, 24 hours, or more.

In other embodiments, at least a portion of the organic solvent can be removed from the plurality of precursor support particles by drying the plurality of precursor support particles under a vacuum in the substantial absence of an atmosphere to produce a plurality of pre-dried support particles. In such embodiment, the plurality of precursor support particles can be at a temperature in a range from 20° C., 50° C., 100° C., or 150° C., to 200° C., 250° C., 300° C., or 340° C. In such embodiment, the plurality of precursor support particles can be dried for a time period in a range from 30 minutes, 1 hour, 1.5 hours, 2 hours, 5 hours, 7 hours, or 10 hours to 15 hours, 17 hours, 20 hours, 24 hours, or more. In some embodiments, the process can optionally include contacting the plurality of pre-dried precursor with a Pt-containing compound and/or a promoter-containing compound and/or an alkali metal element-containing compound.

The plurality of pre-dried support particles can be further dried under a pressure of at least atmospheric pressure to produce the plurality of dried precursor support particles. The plurality of pre-dried precursor support particles can be at a temperature in a range from 20° C., 50° C., 100° C., or 150° C., to 200° C., 250° C., 300° C., or 340° C., when dried under the pressure of at least atmospheric pressure. The plurality of pre-dried precursor support particles can be dried for a time period in a range from 30 minutes, 1 hour, 1.5 hours, 2 hours, 5 hours, 7 hours, or 10 hours to 15 hours, 17 hours, 20 hours, 24 hours, or more. In some embodiments, the pre-dried precursor support particles can be dried under an atmosphere that can be or can include, but is not limited to, oxygen, nitrogen, argon, carbon dioxide, methane, air, or any mixture thereof.

The process can also include heating the plurality of dried precursor support particles to a temperature of at least 350° C., to produce a plurality of calcined support particles. In some embodiments, the process can also include optionally contacting the plurality of calcined support particles with a Pt-containing compound and/or a promoter-containing compound and/or an alkali metal element-containing compound. In some embodiments, the plurality of dried precursor support particles can be heated to a temperature of 350° C., 375° C., 400° C., 425° C., or 450° C., to 475° C., 500° C., 525° C., 550° C., 575° C., 600° C., or more to produce the plurality of calcined support particles. In some embodiments, the plurality of dried precursor support particles can be heated for a time period in a range from 1 minute, 5 minutes, 15 minutes, 30 minutes, 45 minutes, 1 hour, or 2 hours to 3 hours, 4 hours, ≤5 hours, 7 hours, 10 hours, or more to produce the plurality of calcined support particles. In some embodiments, the plurality of dried precursor support particles can be heated under an oxidizing atmosphere, an inert atmosphere, or a reducing atmosphere. In some embodiments, the oxidizing atmosphere can be or can include, but is not limited to air, O₂ enriched air, O₂ depleted air, or any other suitable O₂ containing stream. In some embodiments, the inert atmosphere can be or can include, but is not limited to, Ar, Ne, He, N₂, CO₂, H₂O, or any mixture thereof. In some embodiments, the reducing atmosphere can be or can include, but is not limited to, H₂, CO, CH₄, C₂H₆, C₃H₅, C₂H₄, C₃H₆, steam, or a mixture thereof.

In some embodiments, at least a portion of the organic solvent can be removed from the plurality of precursor support particles by drying the plurality of precursor support particles under a vacuum in the substantial absence of an atmosphere to produce a plurality of pre-dried support particles. In some embodiments, the process can optionally include contacting the plurality of pre-dried precursor with a Pt-containing compound and/or a promoter-containing compound and/or an alkali metal element-containing compound. In such embodiment, the process can also include heating the plurality of pre-dried precursor support particles to a temperature of at least 350° C., to produce the plurality of calcined support particles. As such, in some embodiments, the plurality of pre-dried precursor support particles can be calcined without undergoing the step of producing the dried precursor support particles.

The process for making the catalyst composition can include adding the Pt-containing compound and the promoter-containing compound, and optionally the alkali metal element-containing compound independently during at least one of the steps used to make the catalyst composition. In other words, the Pt-containing compound and the promoter-containing compound, and optionally the alkali metal element-containing compound can be added as an ingredient during preparation of the slurry or gel, contacted with the slurry or gel, contacted with the pre-dried precursor support particles, contacted with the dried precursor support particles, and/or contacted with the calcined support particles.

In at least one embodiment, the Pt-containing compound and the promoter-containing compound and optionally the alkali metal element-containing compound can be added via an incipient wetness impregnation of the plurality of calcined support particles. In such embodiment, the impregnated plurality of calcined support particles can be dried at a temperature in a range from 20° C., 35° C., 50° C., 75° C., or 90° C., to 100° C., 110° C., 125° C., 150° C., 175° C., 200° C., 225° C., 250° C., 275° C., 300° C., 325° C., 340° C., or more for a time period in a range from 15 minutes, 30 minutes 1 hour, 1.5 hours, 2 hours, 2.5 hours, 3, hours, or 4 hours to 5 hours, 6 hours, 8 hours, 10 hours, 12 hours 15 hours or more such the catalyst composition can include the re-calcined support particles.

The impregnated plurality of calcined support particles can be dried under any suitable atmosphere, e.g., an oxidizing atmosphere, an inert atmosphere, a reducing atmosphere, or under a vacuum. The impregnated plurality of calcined support particles can be heated under an oxidizing atmosphere, an inert atmosphere, or a reducing atmosphere. In some embodiments, the oxidizing atmosphere can be or can include, but is not limited to air, O₂ enriched air, O₂ depleted air, or any other suitable O₂ containing stream. In some embodiments, the inert atmosphere can be or can include, but is not limited to, Ar, Ne, He, N₂, CO₂, H₂O, or any mixture thereof. In some embodiments, the reducing atmosphere can be or can include, but is not limited to, H₂, CO, CH₄, C₂H₆, C₃H₅, C₂H₄, C₃H₆, steam, or a mixture thereof.

As noted above, when the Pt-containing compound and the promoter-containing compound are contacted with the plurality of support particles during synthesis of the catalyst composition, e.g., contacted with the pre-dried precursor support particles, the dried precursor support particles, and/or the calcined support particles, the pre-dried precursor support particles, the dried precursor support particles, and/or the calcined support particles can have a surface area ≥300 m²/g, 303 m²/g, ≥305 m²/g, ≥310 m²/g, ≥315 m²/g, 320 m²/g, 325 m²/g, 330 m/g, ≥335 m²/g, ≥340 m²/g, ≥345 m²/g, 350 m²/g, ≥355 m²/g, ≥360 m²/g, ≥365 m/g, ≥370 m²/g, ≥375 m²/g, ≥380 m²/g, ≥385 m²/g, ≥390 m²/g, ≥395 m²/g, ≥400 m²/g, ≥425 m²/g, or 450 m²/g to ≤475 m²/g, 500 m/g, ≤525 m²/g, 550 mg, ≤575 m²/g, ≤600 m²/g, ≥625 m²/g, ≤650 m²/g.

### First Process for Upgrading a Hydrocarbon

The first process for upgrading a hydrocarbon can include contacting a hydrocarbon-containing feed (first hydrocarbon-containing feed) with the catalyst composition that includes Pt and the promoter disposed on the support, where the support includes a mixed Mg/Al metal oxide to effect one or more of dehydrogenation, dehydroaromatization, and dehydrocyclization of at least a portion of the first hydrocarbon-containing feed to produce a coked catalyst composition and an effluent that can include one or more upgraded hydrocarbons and molecular hydrogen. In some embodiments, the support can have a surface area ≥300 m²/g when contacted with the first hydrocarbon-containing feed. In other embodiments, the support can have a surface area <300 m²/g when contacted with the first hydrocarbon-containing feed. However, when the support has a surface area <300 m²/g when contacted with the first hydrocarbon-containing feed, during synthesis of the catalyst composition, when the Pt-containing compound and the promoter-containing compound are contacted with the support particles, e.g., the pre-dried precursor support particles, the dried precursor support particles, and/or the calcined support particles, the support particles can have a surface area ≥300 m²/g. Further processing of the catalyst composition after the addition of the Pt-containing compound and the promoter-containing compound, e.g., additional calcination, can reduce the surface area of the support to <300 m²/g before the catalyst composition contacts the first hydrocarbon containing feed.

The catalyst composition and the first hydrocarbon-containing feed can be contacted with one another within any suitable environment such as one or more reaction or conversion zones disposed within one or more reactors to produce the effluent and the coked catalyst composition. The reaction or conversion zone can be disposed or otherwise located within one or more fixed bed reactors, one or more fluidized or moving bed reactors, one or more reverse flow reactors, or any combination thereof.

The first hydrocarbon-containing feed and the catalyst composition can be contacted at a temperature in a range from 300° C., 350° C., 400° C., 450° C., 500° C., 550° C., 600° C., 620° C. 650° C., 660° C., 670° C., 680° C., 690° C., or 700° C., to 725° C., 750° C., 760° C., 780° C., 800° C., 825° C., 850° C., 875° C., or 900° C. In some embodiments, the first hydrocarbon-containing feed and the catalyst composition can be contacted at a temperature of at least 620° C., at least 650° C., at least 660° C., at least 670° C., at least 680° C., at least 690° C., or at least 700° C., to 725° C., 750° C., 760° C., 780° C., 800° C., 825° C., 850° C., 875° C., or 900° C. The first hydrocarbon-containing feed can be introduced into the reaction or conversion zone and contacted with the catalyst composition therein for a time period of ≤3 hours, ≤2.5 hours, ≤2 hours, ≤1.5 hours, ≤1 hour, ≤45 minutes, ≤30 minutes, ≤20 minutes, ≤10 minutes, ≤5 minutes, ≤1 minute, ≤30 seconds, ≤10 seconds, ≤5 seconds, or ≤1 second or ≤0.5 second. In some embodiments, the first hydrocarbon-containing feed can be contacted with the catalyst composition for a time period in a range from 0.1 seconds, 0.5 seconds, 0.7 seconds, 1 second, 30 second, 1 minute, 5 minutes, or 10 minutes to 30 minutes, 50 minutes, 70 minutes, 1.5 hours, 2 hours, or 3 hours.

The first hydrocarbon-containing feed and the catalyst composition can be contacted under a hydrocarbon partial pressure of at least 20 kPa-absolute, where the hydrocarbon partial pressure is the total partial pressure of any C₂-C₁₆ alkanes and any C₈-C₁₆ alkyl aromatics in the first hydrocarbon-containing feed. In some embodiments, the hydrocarbon partial pressure during contact of the first hydrocarbon-containing feed and the catalyst composition can be in a range from 20 kPa-absolute, 50 kPa-absolute, 100 kPa-absolute, at least 150 kPa, at least 200 kPa 300 kPa-absolute, 500 kPa-absolute, 750 kPa-absolute, or 1,000 kPa-absolute to 1,500 kPa-absolute, 2.500 kPa-absolute, 4.000 kPa-absolute, 5.000 kPa-absolute, 7.000 kPa-absolute, 8,500 kPa-absolute, or 10.000 kPa-absolute, where the hydrocarbon partial pressure is the total partial pressure of any C₂-C₁₆ alkanes and any C₈-C₁₆ alkyl aromatics in the first hydrocarbon-containing feed. In other embodiments, the hydrocarbon partial pressure during contact of the first hydrocarbon-containing feed and the catalyst composition can be in a range from 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute, where the hydrocarbon partial pressure is the total partial pressure of any C₂-C₁₆ alkanes and any C₈-C₁₆ alkyl aromatics in the first hydrocarbon-containing feed.

In some embodiments, the first hydrocarbon-containing feed can include at least 60 vol %, at least 65 vol %, at least 70 vol %, at least 75 vol %, at least 80 vol %, at least 85 vol %, at least 90 vol %, at least 95 vol %, or at least 99 vol % of a single C₂-C₁₆ alkane, e.g., propane, based on a total volume of the first hydrocarbon-containing feed. The first hydrocarbon-containing feed and the catalyst composition can be contacted under a single C₂-C₁₆ alkane, e.g., propane, pressure of at least 20 kPa-absolute, at least 50 kPa-absolute, at least 100 kPa-absolute, at least 150 kPa-absolute, at least 250 kPa-absolute, at least 300 kPa-absolute, at least 400 kPa-absolute, at least 500 kPa-absolute, or at least 1.000 kPa-absolute.

The first hydrocarbon-containing feed can be contacted with the catalyst composition within the reaction or conversion zone at any weight hourly space velocity (WHSV) effective for carrying out the upgrading process. In some embodiments, the WHSV can be 0.01 hr⁻¹, 0.1 hr⁻¹, 1 hr⁻¹, 2 hr⁻¹, 5 hr⁻¹, 10 hr⁻¹, 20 hr⁻¹, 30 hr⁻¹, or 50 hr⁻¹ to 100 hr⁻¹, 250 hr⁻¹, 500 hr⁻¹, or 1,000 hr⁻¹. In some embodiments, when the hydrocarbon upgrading process includes a fluidized or otherwise moving catalyst composition, a ratio of the catalyst composition circulation mass flow rate to a combined amount of any C₂-C₁₆ alkanes and any C₈-C₁₆ alkyl aromatics mass flow rate can be in a range from 1, 3, 5, 10, 15, 20, 25, 30, or 40 to 50, 60, 70, 80, 90, 100, 110, 125, or 150 on a weight to weight basis.

When the activity of the coked catalyst composition decreases below a desired minimum amount, the coked catalyst composition or at least a portion thereof can be subjected to a regeneration process to produce a regenerated catalyst composition. More particularly, the coked catalyst composition can be contacted with one or more oxidants to effect combustion of at least a portion of the coke to produce a regenerated catalyst composition lean in coke and a combustion gas. Regeneration of the coked catalyst composition can occur within the reaction or conversion zone or within a combustion zone that is separate and apart from the reaction or conversion zone, depending on the particular reactor configuration, to produce the regenerated catalyst composition. For example, regeneration of the coked catalyst composition can occur within the reaction or conversion zone when a fixed bed or reverse flow reactor is used, or within a separate combustion zone that can be separate and apart from the reaction or conversion zone when a fluidized bed reactor or other circulating or fluidized type reactor is used.

In some embodiments the process can optionally include contacting at least a portion of the regenerated catalyst composition with a reducing gas to produce a regenerated and reduced catalyst composition. An additional quantity of the first hydrocarbon-containing feed can be contacted with at least a portion of the regenerated catalyst composition and/or at least a portion of any regenerated and reduced catalyst composition to produce a re-coked catalyst composition and additional effluent. Reduction of the regenerated catalyst composition can occur within the reaction or conversion zone, within the regeneration zone, or within a reduction zone that is separate and apart from the reaction or conversion zone and the regeneration zone, depending on the particular reactor configuration, to produce the regenerated and reduced catalyst composition. For example, reduction of the regenerated catalyst composition can occur within the reaction or conversion zone when a fixed bed or reverse flow reactor is used, or within a separate reduction zone that can be separate and apart from the reaction or conversion zone and the regeneration zone when a fluidized bed reactor or other circulating or fluidized type reactor is used.

In some embodiments, a cycle time from contacting the first hydrocarbon-containing feed with the catalyst composition to contacting the additional quantity of the first hydrocarbon-containing feed with the regenerated catalyst composition can be ≤5 hours. The first cycle begins upon contact of the catalyst composition with the first hydrocarbon-containing feed, followed by contact with at least the oxidative gas to produce the regenerated catalyst composition or at least the oxidative gas and the optional reducing gas to produce the regenerated catalyst composition, and the first cycle ends upon contact of the regenerated catalyst composition with the additional quantity of the first hydrocarbon-containing feed. If one or more additional feeds (described in more detail below) are utilized between flows of the first hydrocarbon-containing feed and the oxidative gas, between the oxidative gas and the reducing gas (if used), between the oxidative gas and the additional quantity of the first hydrocarbon-containing feed, and/or between the reducing gas (if used) and the additional quantity of the first hydrocarbon-containing feed, the period of time such stripping gas(es) is/are utilized would be included in the period included in the cycle time. As such, the cycle time from contacting the first hydrocarbon-containing feed with the catalyst composition to the contacting the additional quantity of the first hydrocarbon-containing feed with the regenerated catalyst composition, in some embodiments, can be ≤5 hours, ≤4 hours, ≤3 hours, ≤2 hours, ≤1 hour, ≤50 minutes, ≤45 minutes, ≤30 minutes, ≤15 minutes, ≤10 minutes, ≤5 minutes, ≤1 minute, ≤30 seconds, or ≤10 seconds.

The oxidant can be or can include, but is not limited to, O₂, O₃, CO₂, H₂O, or a mixture thereof. In some embodiments, an amount of oxidant in excess of that needed to combust 100% of the coke on the catalyst composition can be used to increase the rate of coke removal from the catalyst composition, so that the time needed for coke removal can be reduced and lead to an increased yield in the upgraded product produced within a given period of time. The use of pure O₂ as an oxidant can facilitate the capturing and sequestration of CO₂ made during combustion in one or more downstream CO₂ recovery systems.

The coked catalyst composition and oxidant can be contacted with one another at a temperature in a range from 500° C., 550° C., 600° C., 650° C., 700° C., 750° C., or 800° C., to 900° C. 950° C., 1,000° C., 1,050° C., or 1,100° C., to produce the regenerated catalyst composition. In some embodiments, the coked catalyst composition and oxidant can be contacted with one another at a temperature in a range from 500° C., to 1,100° C., 600° C., to 1,000° C., 650° C., to 950° C., 700′C to 900° C., or 750° C., to 850° C., to produce the regenerated catalyst composition.

The coked catalyst composition and oxidant can be contacted with one another for a time period of ≤2 hours, ≤1 hour, ≤30 minutes, ≤10 minutes, ≤5 minutes, ≤1 min, ≤30 seconds, ≤10 seconds, ≤5 seconds, or ≤1 second. For example, the coked catalyst composition and oxidant can be contacted with one another for a time period in a range from 2 seconds to 2 hours. In some embodiments, the coked catalyst composition and oxidant can be contacted for a time period sufficient to remove ≥50 wt %, ≥75 wt %, or ≥90 wt % or ≥99% of any coke disposed on the catalyst composition.

In some embodiments, the time period the coked catalyst composition and oxidant contact one another can be less than the time period the catalyst composition contacts the first hydrocarbon-containing feed to produce the effluent and the coked catalyst composition. For example, the time period the coked catalyst composition and oxidant contact one another can be at least 90%, at least 60%, at least 30%, or at least 10% less than the time period the catalyst composition contacts the first hydrocarbon-containing feed to produce the effluent. In other embodiments, the time period the coked catalyst composition and oxidant contact one another can be greater than the time period the catalyst composition contacts the first hydrocarbon-containing feed to produce the effluent and the coked catalyst composition. For example, in some embodiments, the coked catalyst composition and oxidant can contact one another for a time period that can be at least 50%, at least 100%, at least 300%, at least 500%, at least 1,000%, at least 10,000%, at least 30,000%, at least 50,000%, at least 75,000%, at least 100,000%, at least 250,000%, at least 500,000%, at least 750,000%, at least 1,000,000%, at least 1,250,000%, at least 1,500,000%, or at least 1,800,000% greater than the time period the catalyst composition contacts the first hydrocarbon-containing feed to produce the effluent.

The coked catalyst composition and oxidant can be contacted with one another under an oxidant partial pressure in a range from 20 kPa-absolute, 50 kPa-absolute, 100 kPa-absolute, 300 kPa-absolute, 500 kPa-absolute, 750 kPa-absolute, or 1,000 kPa-absolute to 1,500 kPa-absolute, 2.500 kPa-absolute, 4.000 kPa-absolute, 5.000 kPa-absolute, 7,000 kPa-absolute, 8,500 kPa-absolute, or 10,000 kPa-absolute. In other embodiments, the oxidant partial pressure during contact with the coked catalyst composition can be in a range from 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute to produce the regenerated catalyst composition.

Without wishing to be bound by theory, it is believed that at least a portion of the Group 8-10 element present in/on the coked catalyst composition can be agglomerated as compared to the catalyst composition prior to contact with the first hydrocarbon-containing feed. It is believed that during combustion of at least a portion of the coke on the coked catalyst composition that at least a portion of the Group 8-10 element can be re-dispersed about the support of the catalyst composition. Re-dispersing at least a portion of any agglomerated Group 8-10 element can improve the stability of the catalyst composition over many cycles.

In some embodiments, at least a portion of the Group 8-10 element in the regenerated catalyst composition can be at a higher oxidized state as compared to the Group 8-10 element in the catalyst composition contacted with the first hydrocarbon-containing feed and as compared to the Group 8-10 element in the coked catalyst composition. As such, as noted above, in some embodiments the process can optionally include contacting at least a portion of the regenerated catalyst composition with a reducing gas to produce a regenerated and reduced catalyst composition. Suitable reducing gases (reducing agent) can be or can include, but are not limited to, H₂, CO, CH₄, C₂H₆, C₃H₈, C₂H₄, C₃H₆, steam, or a mixture thereof. In some embodiments, the reducing agent can be mixed with an inert gas such as Ar, Ne, He, N₂, CO₂. H₂O or a mixture thereof. In such embodiments, at least a portion of the Group 8-10 element in the regenerated and reduced catalyst composition can be reduced to a lower oxidation state, e.g., the elemental state, as compared to the Group 8-10 element in the regenerated catalyst composition. In this embodiment, the additional quantity of the first hydrocarbon-containing feed can be contacted with at least a portion of the regenerated catalyst composition and/or at least a portion of the regenerated and reduced catalyst composition.

In some embodiments, the regenerated catalyst composition and the reducing gas can be contacted at a temperature in a range from 400° C., 450° C., 500° C., 550° C., 600° C., 620° C., 650° C., or 670° C., to 720° C., 750° C., 800° C., or 900° C. The regenerated catalyst composition and the reducing gas can be contacted for a time period in a range from 1 second, 5 seconds, 10 seconds, 20 seconds, 30 seconds, or 1 minute to 10 minutes, 30 minutes, or 60 minutes. The regenerated catalyst composition and reducing gas can be contacted at a reducing agent partial pressure of 20 kPa-absolute, 50 kPa-absolute, or 100 kPa-absolute, 300 kPa-absolute, 500 kPa-absolute, 750 kPa-absolute, or 1,000 kPa-absolute to 1.500 kPa-absolute, 2.500 kPa-absolute, 4,000 kPa-absolute, 5,000 kPa-absolute, 7,000 kPa-absolute, 8,500 kPa-absolute, or 10,000 kPa-absolute. In other embodiments, the reducing agent partial pressure during contact with the regenerated catalyst composition can be in a range from 20 kPa-absolute, 50 kPa-absolute, 100 kPa-absolute, 150 kPa-absolute, 200 kPa-absolute, 250 kPa-absolute, or 300 kPa-absolute to 500 kPa-absolute, 600 kPa-absolute, 700 kPa-absolute, 800 kPa-absolute, 900 kPa-absolute, or 1,000 kPa-absolute to produce the regenerated catalyst composition.

At least a portion of the regenerated catalyst composition, the regenerated and reduced catalyst composition, new or fresh catalyst composition, or a mixture thereof can be contacted with an additional quantity of the first hydrocarbon-containing feed within the reaction or conversion zone to produce additional effluent and additional coked catalyst composition. As noted above, in some embodiments, the cycle time from the contacting the first hydrocarbon-containing feed with the catalyst composition to the contacting the additional quantity of the first hydrocarbon-containing feed with at least a portion of the regenerated catalyst composition, and/or the regenerated and reduced catalyst composition, and optionally with new or fresh catalyst composition can be ≤5 hours, ≤4 hours, ≤3 hours, ≤2 hours, ≤1 hour, ≤50 minutes, ≤45 minutes, ≤30 minutes, ≤15 minutes, ≤10 minutes, ≤5 minutes, ≤1 minute, ≤30 seconds, or ≤10 seconds.

In some embodiments, as noted above, one or more additional feeds, e.g., one or more sweep fluids, can be utilized between flows of the first hydrocarbon-containing feed and the oxidant, between the oxidant and the optional reducing gas if used, between the oxidant and the additional first hydrocarbon-containing feed, and/or between the reducing gas and the additional first hydrocarbon-containing feed. The sweep fluid can, among other things, purge or otherwise urge undesired material from the reactors, such as non-combustible particulates including soot. In some embodiments, the additional feed(s) can be inert under the dehydrogenation, dehydroaromatization, and dehydrocyclization, combustion, and/or reducing conditions. Suitable sweep fluids can be or can include, but are not limited to, N₂, He, Ar, CO₂, H₂O, CO₂, CH₄, or a mixture thereof. In some embodiments, if the process utilizes a sweep fluid the duration or time period the sweep fluid is used can be in a range from 1 second, ≤5 seconds, 10 seconds, 20 seconds, 30 seconds, or 1 minute to 10 minutes, 30 minutes, or 60 minutes.

In some embodiments, the catalyst composition can remain sufficiently active and stable after many cycles, e.g., at least 15, at least 20, at least 30, at least 40, at least 50, at least 60, at least 70, at least 100 cycles, at least 125 cycles, at least 150 cycles, at least 175 cycles, or at least 200 cycles with each cycle time lasting for ≤5 hours, ≤4 hours, ≤3 hours, ≤2 hours, ≤1 hour, ≤50 minutes, ≤45 minutes, 30 minutes, 15 minutes, 10 minutes, ≤5 minutes, ≤1 minute, 30 seconds, or 10 seconds. In some embodiments, the cycle time can be from 5 seconds, 30 seconds, 1 minute or 5 minutes to 10 minutes, 20 minutes, 30 minutes, 45 minutes, 50 minutes, 70 minutes, 2 hours, 3 ours, 4 hours, or 5 hours. In some embodiments, after the catalyst performance stabilizes (sometimes the first few cycles can have a relatively poor or a relatively good performance, but the performance can eventually stabilize), the process can produce a first upgraded hydrocarbon product yield, e g., propylene when the first hydrocarbon-containing feed includes propane, at an upgraded hydrocarbon selectivity, e.g., propylene, of ≥75%, ≥80%, ≥85%, ≥90%, ≥93%, or ≥95% when initially contacted with the first hydrocarbon-containing feed, and can have a second upgraded hydrocarbon product yield upon completion of the last cycle (at least 15 cycles total) that can be at least 90%, at least 93%, at least 95%, at least 97%, at least 98%, at least 99%, at least 99.5%, or at least 100% of the first upgraded hydrocarbon product yield at an upgraded hydrocarbon selectivity, e.g., propylene, of ≥75%, ≥80%, ≥85%, or ≥90%, ≥93%, or ≥95%.

In some embodiments, when the first hydrocarbon-containing feed includes propane and the upgraded hydrocarbon includes propylene, contacting the first hydrocarbon-containing feed with the catalyst composition can produce a propylene yield of ≥52%, ≥53%, 55%, ≥57%, ≥60%, ≥62%, ≥63%, ≥64%, ≥65%, or 66% at a propylene selectivity of ≥75%, ≥80%, ≥85%, ≥90%, ≥93%, or ≥95% for at least 15, at least 20, at least 30, at least 40, at least 50, at least 60, at least 70, at least 100 cycles, at least 125 cycles, at least 150 cycles, at least 175 cycles, or at least 200 cycles. In other embodiments, when the first hydrocarbon-containing feed includes at least 70 vol % of propane, based on a total volume of the first hydrocarbon-containing feed, is contacted under a propane partial pressure of at least 20 kPa-absolute, a propylene yield of ≥52%, ≥53%, ≥55%, 57%, ≥60%, ≥62%, ≥63%, ≥64%, ≥65%, or ≥66% at a propylene selectivity of ≥75%, ≥80%, ≥85%, ≥90%, ≥93%, or ≥95% can be obtained for at least 15, at least 20, at least 30, at least 40, at least 50, at least 60, at least 70, at least 100 cycles, at least 125 cycles, at least 150 cycles, at least 175 cycles, or at least 200 cycles. It is believed that the propylene yield can be further increased to ≥67%, ≥68%, ≥70%, ≥72%, ≥75%, ≥77%, ≥80%, or ≥82% at a propylene selectivity of ≥75%, ≥80%, 85%, 90%, ≥93%, or ≥95% for ≥15 cycles, ≥20 cycles, ≥30 cycles, ≥40 cycles, 50 cycles, ≥60 cycles, ≥70 cycles, ≥100 cycles, ≥125 cycles, ≥150 cycles, ≥175 cycles, or 200 cycles by further optimizing the composition of the support and/or adjusting one or more process conditions. In some embodiments, the propylene yield can be obtained when the catalyst composition is contacted with the first hydrocarbon-containing feed at a temperature of >620° C., ≥630° C., ≥640° C., ≥650° C., ≥655° C. ≥660° C., ≥670° C., ≥680° C., ≥690° C., ≥700° C. or ≥750° C. for 15 cycles, 20 cycles, ≥30 cycles, 40 cycles, ≥50 cycles, ≥60 cycles, ≥70 cycles, 100 cycles, 125 cycles, ≥150 cycles, ≥175 cycles, or ≥200 cycles.

In some embodiments, when a fluidized bed reactor or other circulating or fluidized type reactor is used, the catalyst composition can be introduced into any location or combination of locations of the reactor system. In some embodiments, the catalyst composition can be introduced into the reaction or conversion zone, the regeneration zone, if present, the reduction zone, any location located between any two of the zones or any combination thereof.

Systems suitable for carrying out the processes disclosed herein can include systems that are well-known in the art such as the fixed bed reactors disclosed in WO Publication No. WO2017078894; the fluidized riser reactors and/or downer reactors disclosed in U.S. Pat. Nos. 3,888,762; 7,102,050; 7,195,741; 7,122,160; and 8,653,317; and U.S. Patent Application Publication Nos. 2004/0082824; 2008/0194891; and the reverse flow reactors disclosed in U.S. Pat. No. 8,754,276; U.S. Patent Application Publication No. 2015/0065767; and WO Publication No. WO2013169461.

### First Hydrocarbon-Containing Feed

The first hydrocarbon-containing feed can be or can include, but is not limited to, one or more alkane hydrocarbons, e.g., C₂-C₁₆ linear or branched alkanes and/or C₄-Cᵣ₆ cyclic alkanes, and/or one or more alkyl aromatic hydrocarbons, e.g., C₈-C₁₆ alkyl aromatics. In some embodiments, the first hydrocarbon-containing feed can optionally include 0.1 vol % to 50 vol % of steam, based on a total volume of any C₂-C₁₆ alkanes and any C₅-C₁₆ alkyl aromatics in the first hydrocarbon-containing feed in other embodiments, the first hydrocarbon-containing feed can include <0.1 vol % of steam or can be free of steam, based on the total volume of any C₂-C₁₆ alkanes and any C₅-C₁₆ alkyl aromatics in the first hydrocarbon-containing feed.

The C₂-C₁₆ alkanes can be or can include, but are not limited to, ethane, propane, n-butane, isobutane, n-pentane, isopentane, n-hexane, 2-methylpentane, 3-methylpentane, 2,2-dimethylbutane, n-heptane, 2-methylhexane, 2,2,3-trimethylbutane, cyclopentane, cyclohexane, methylcyclopentane, ethylcyclopentane, n-propylcyclopentane, 1,3-dimethylcyclohexane, or a mixture thereof. For example, the first hydrocarbon-containing feed can include propane, which can be dehydrogenated to produce propylene, and/or isobutane, which can be dehydrogenated to produce isobutylene. In another example, the first hydrocarbon-containing feed can include liquid petroleum gas (LP gas), which can be in the gaseous phase when contacted with the catalyst composition. In some embodiments, the hydrocarbon in the first hydrocarbon-containing feed can be composed of substantiallu a single alkane such as propane. In some embodiments, the first hydrocarbon-containing feed can include ≥50 mol %, ≥75 mol %, ≥95 mol %, ≥98 mol %, or ≥99 mol % of a single C₂-C₁₆ alkane, e.g., propane, based on total moles of all hydrocarbons in the first hydrocarbon-containing feed. In some embodiments, the first hydrocarbon-containing feed can include at least 50 vol %, at least 55 vol %, at least 60 vol %, at least 65 vol %, at least 70 vol %, at least 75 vol %, at least 80 vol %, at least 85 vol %, at least 90 vol %, at least 95 vol %, at least 97 vol %, or at least 99 vol % of a single C₂-C₁₆ alkane, e.g., propane, based on a total volume of the first hydrocarbon-containing feed.

The C₈-C₁₆ alkyl aromatics can be or can include, but are not limited to, ethylbenzene, propylbenzene, butylbenzene, one or more ethyl toluenes, or a mixture thereof. In some embodiments, the first hydrocarbon-containing feed can include ≥50 mol %, ≥75 mol %, % 95 mol %, ≥98 mol %, or ≥99 mol % of a single C₈-C₁₆ alkyl aromatic, e.g., ethylbenzene, based on a total weight of all hydrocarbons in the first hydrocarbon-containing feed. In some embodiments, the ethylbenzene can be dehydrogenated to produce styrene. As such, in some embodiments, the first process for upgrading a hydrocarbon disclosed herein can include propane dehydrogenation, butane dehydrogenation, isobutane dehydrogenation, pentane dehydrogenation, pentane dehydrocyclization to cyclopentadiene, naphtha reforming, ethylbenzene dehydrogenation, ethyltoluene dehydrogenation, and the like.

In some embodiments, the first hydrocarbon-containing feed can be diluted, e.g., with one or more diluents such as one or more inert gases. Suitable inert gases can be or can include, but are not limited to, Ar, Ne, He, N₂, CO₂, CH₄, or a mixture thereof. If the hydrocarbon containing-feed includes a diluent, the first hydrocarbon-containing feed can include 0.1 vol %, 0.5 vol %, 1 vol %, or 2 vol % to 3 vol %, 8 vol %, 16 vol %, or 32 vol % of the diluent, based on a total volume of any C₂-C₁₆ alkanes and any C₅-C₁₆ alkyl aromatics in the first hydrocarbon-containing feed.

In some embodiments, the first hydrocarbon-containing feed can also include H₂. In some embodiments, when the first hydrocarbon-containing feed includes H₂, a molar ratio of the H₂ to a combined amount of any C₂-C₁₆ alkane and any C₈-C₁₆ alkyl aromatic can be in a range from 0.1, 0.3, 0.5, 0.7, or 1 to 2, 3, 4, 5, 6, 7, 8, 9, or 10. In other embodiments, H₂ can be introduced into the reactor system as a feed separate and apart from the first hydrocarbon-containing feed.

In some embodiments, first hydrocarbon-containing feed and the environment within the reactor system can be substantially free of any steam, e.g., ≤0.1 vol % of steam, based on a total volume of any C₂-C₁₆ alkanes and any C₅-C₁₆ alkyl aromatics in the first hydrocarbon-containing feed. In other embodiments, the first hydrocarbon-containing feed can include steam and/or steam can be introduced into the reactor system as a feed separate and apart from the first hydrocarbon-containing feed. For example, the first hydrocarbon-containing feed or, if introduced separate from the first hydrocarbon-containing feed, the environment within the reactor system can include 0.1 vol %, 0.3 vol %, 0.5 vol %, 0.7 vol %, 1 vol %, 3 vol %, or 5 vol % to 10 vol %, 15 vol %, 20 vol %, 25 vol %, 30 vol %, 35 vol %, 40 vol %, 45 vol %, or 50 vol % of steam, based on a total volume of any C₂-C₁₆ alkanes and any C₈-C₁₆ alkyl aromatics in the first hydrocarbon-containing feed. In other embodiments, the first hydrocarbon-containing feed or, if introduced separate from the first hydrocarbon-containing feed, the environment within the reactor system can include ≤50 vol %, ≤45 vol %, ≤40 vol %, ≤35 vol %, ≤30 vol %, ≤25 vol %, ≤20 vol %, or 15 vol % of steam, based on a total volume of any C₂-C₁₆ alkanes and any C₈-C₁₆ alkyl aromatics in the first hydrocarbon-containing feed. In other embodiments, the first hydrocarbon-containing feed or, if introduced separate from the first hydrocarbon-containing feed, the environment within the reactor system can include at least 1 vol %, at least 3 vol %, at least 5 vol %, at least 10 vol %, at least 15 vol %, at least 20 vol %, at least 25 vol %, or at least 30 vol % of steam, based on a total volume of any C₂-C₁₆ alkanes and any C₈-C₁₆ alkyl aromatics in the first hydrocarbon-containing feed.

In some embodiments, the first hydrocarbon-containing feed can include sulfur or sulfur can be introduced as a feed separate and apart from the first hydrocarbon-containing feed. For example, the first hydrocarbon-containing feed can include sulfur in a range from 0.5 ppm, 1 ppm, 5 ppm, 10 ppm, 20 ppm 30 ppm, 40 ppm, 50 ppm, 60 ppm, 70 ppm, or 80 ppm to 100 ppm, 150 ppm, 200 ppm, 300 ppm, 400 ppm, or 500 ppm. In other embodiments, the first hydrocarbon-containing feed can include sulfur in a range from 1 ppm to 10 ppm, 10 ppm to 20 ppm, 20 ppm to 50 ppm, 50 ppm to 100 ppm, or 100 ppm to 500 ppm. The sulfur, if present in the first hydrocarbon-containing feed, can be or can include, but is not limited to, H₂S, dimethyl disulfide, as one or more mercaptans, or any mixture thereof.

In some embodiments, the first hydrocarbon-containing feed and the environment within the reaction or conversion zone can be substantially free or free of molecular oxygen. In some embodiments, the first hydrocarbon-containing feed can include ≤5 mol %, ≤3 mol %, or ≤1 mol % of molecular oxygen (O2). It is believed that providing a first hydrocarbon-containing feed substantially-free of molecular oxygen substantially prevents oxidative reactions that would otherwise consume at least a portion of the alkane and/or the alkyl aromatic in first hydrocarbon-containing feed.

### Recovery and Use of the First Upgraded Hydrocarbon

In some embodiments, the first upgraded hydrocarbon in the effluent can include at least one upgraded hydrocarbon, e.g., an olefin, water, unreacted hydrocarbons, molecular hydrogen, etc. The upgraded hydrocarbon can be recovered or othenvise obtained via any convenient process, e.g., by one or more conventional processes. One such process can include cooling and/or compressing the effluent to condense at least a portion of any water and any heavy hydrocarbon that may be present, leaving the olefin and at least a portion of any unreacted alkane or alkyl aromatic primarily in the vapor phase. Olefin and unreacted alkane or alkyl aromatic hydrocarbons can then be removed from the reaction product in one or more separator drums. For example, one or more splitters or distillation columns can be used to separate the dehydrogenated product from the unreacted first hydrocarbon-containing feed.

In some embodiments, a recovered olefin, e.g., propylene, can be used for producing polymer, e.g., recovered propylene can be polymerized to produce polymer having segments or units derived from the recovered propylene such as polypropylene, ethylene-propylene copolymer, etc. Recovered isobutene can be used, e.g., for producing one or more of: an oxygenate such as methyl tert-butyl ether, fuel additives such as diisobutene, synthetic elastomeric polymer such as butyl rubber, etc.

### A Second Process for Upgrading a Hydrocarbon

The second process for upgrading a hydrocarbon can include contacting a second hydrocarbon-containing feed with the catalyst composition that includes Pt and the promoter disposed on the support, where the support includes a mixed Mg/Al metal oxide to effect reforming of at least a portion of the second hydrocarbon-containing feed to produce a coked catalyst composition and an effluent that can include carbon monoxide and molecular hydrogen. In some embodiments, the support can have a surface area ≥300 m²/g when contacted with the first hydrocarbon-containing feed. In other embodiments, the support can have a surface area <300 m²/g when contacted with the first hydrocarbon-containing feed. However, when the support has a surface area <300 m²/g when contacted with the first hydrocarbon-containing feed, during synthesis of the catalyst composition, when the Pt-containing compound and the promoter-containing compound are contacted with the support particles, e.g., the pre-dried precursor support particles, the dried precursor support particles, and/or the calcined support particles, the support particles can have a surface area ≥300 m²/g. Further processing of the catalyst composition after the addition of the Pt-containing compound and the promoter-containing compound, e.g., additional calcination, can reduce the surface area of the support to <300 m²/g before the catalyst composition contacts the first hydrocarbon containing feed.

The catalyst composition and the second hydrocarbon-containing feed can be contacted with one another within any suitable environment such as one or more reaction or conversion zones disposed within one or more reactors to produce the effluent and the coked catalyst composition. The reaction or conversion zone can be disposed or otherwise located within one or more fixed bed reactors, one or more fluidized or moving bed reactors, one or more reverse flow reactors, or any combination thereof. For clarity and ease of description, the reforming reaction will be discussed in the context of a fluidized bed reactor, but it should be understood that fixed bed reactors, reverse flow or moving bed reactors, or any other reactor can be used to carry out the reforming of the second hydrocarbon-containing feed.

The reforming reaction can be used to produce reformed hydrocarbons via a continuous reaction process or a discontinuous reaction process. In some embodiments, the reaction process can include a reforming step, e.g., an endothermic reaction, and a regeneration step, e.g., an exothermic reaction, that operate continuously while the fluidized catalyst is transported in-between the reforming zone and regeneration zone of the reactor. The endothermic reaction can include hydrocarbon reforming in the presence of the catalyst composition. Fresh hydrocarbon and regenerated fluidized catalyst composition can enter the reforming zone. After spending some time in the reforming zone, the hydrocarbon can be at least partially converted to a reforming product that can exit the reforming zone together with the spent catalyst composition. The reforming product and unreacted feed can be separated from the spent catalyst composition by one or more separating devices. While the reforming product and unreacted feed from the separating devices go downstream for further purification, the spent catalyst composition can be sent to the regeneration zone for regeneration. The exothermic regeneration reaction can be the reaction of an oxidant and, optionally a fuel, under combustion conditions to produce a regenerated catalyst composition and a flue gas. After regeneration, the regenerated catalyst composition can be separated from the flue gas by one or more separating devices and can be transported back to the reforming zone, joining more hydrocarbon feed to enter the reforming zone to initiate more reforming reaction. The reforming step can convert CO₂ and/or H₂O and hydrocarbons, e.g., CH₄, to a synthesis gas that includes H₂ and CO. The regeneration step can combust reactants, e.g., coke disposed on the spent catalyst composition and/or the optional fuel and an oxidant, to generate heat that heats up the regenerated catalyst composition that can provide heat that can be used to drive the reforming reaction. In some embodiments, the catalyst composition can be heated to an average temperature in a range of from 600° C., 700° C., or 800° C., to 1,000° C., 1.300° C., or 1,600° C. during the regeneration step.

Illustrative fuels can be or can include, but are not limited to, hydrocarbons, e.g., methane, ethane, propane, butane, pentane, or hydrocarbon containing streams, e.g., natural gas, molecular hydrogen, fuel oil, heavy fuel oil, gasoline, diesel, kerosene, distillate, and/or other combustible compounds. The oxidant can be or can include O₂. In some embodiments, the oxidant can be or can include air, O₂ enriched air, O₂ depleted air, or any other suitable O₂ containing stream.

The regeneration of the catalyst composition can correspond to removal of coke from the particles in the catalyst composition. In some embodiments, during reforming, a portion of the feed introduced into the reforming zone can form coke. This coke can potentially block access to the catalytic sites (such as metal sites) of the catalyst composition. During regeneration at least a portion of the coke generated during reforming can be removed as CO or CO₂. The regeneration of the catalyst composition can also correspond to re-dispersion of any agglomerated active phase of the catalyst such as the Group 8-10 element.

### Second Hydrocarbon-Containing Feed

The second hydrocarbon-containing feed can be or can include, but is not limited to, one or more reformable C₁-C₁₆ hydrocarbons such as alkanes, alkenes, cycloalkanes, alkylaromatics, or any mixture thereof. In some embodiments, the second hydrocarbon-containing feed can be or can include methane, ethane, propane, butane, pentane, or a mixture thereof. In some embodiments, the second hydrocarbon-containing feed can be exposed to the catalyst composition under a pressure of less than 35 kPag. For example, the second hydrocarbon-containing feed can be exposed to the catalyst composition under a pressure in a range of from 0.7 kPag, 2 kPag, 3.5 kPag, 5 kPag, or 10 kPag to 15 kPag, 20 kPag, 25 kPag, or 30 kPag. In other embodiments, the second hydrocarbon-containing feed can be exposed to the catalyst composition under a pressure in a range of from 35 kPag to 15 MPag. In still other embodiments, the second hydrocarbon-containing feed can be exposed to the catalyst composition under a pressure in a range of from 0.7 kPag, 2 kPag, 5 kPag, 20 kPag, 35 kPag, 50 kPag, or 100 kPag to 200 kPag, 1 MPag, 3 MPag, 5 MPag, 10 MPag, or 15 MPag. In still other embodiments, the second hydrocarbon-containing feed can be exposed to the catalyst composition under a pressure of less than 2.8 MPag, less than 2.5 MPag, less than 2.2 MPag, or less than 2 MPag.

The reforming reaction of the second hydrocarbon-containing feed, e.g., CH₄, can occur in the presence of H₂O (steam-reforming), in the presence of CO₂ (dry-reforming), or in the presence of both H₂O and CO₂ (bi-reforming). Examples of stoichiometry for steam, dry, and bi-reforming of CH₄ are shown in equations (1)-(3).

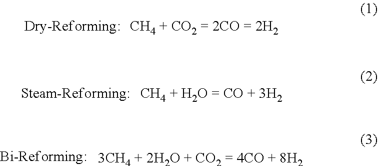

As shown in equations (1)-(3), dry reforming can produce lower ratios of H₂ to CO than steam reforming. Reforming reactions performed with only steam can generally produce a synthesis gas having a H₂:CO molar ratio of around 3, such as 2.5 to 3.5. In contrast, reforming reactions performed with only CO₂ can generally produce a synthesis gas having a H₂:CO molar ratio of roughly 1 or even lower. By using a combination of CO₂ and H₂O during reforming, the reforming reaction can be controlled to generate a wide variety of H₂ to CO ratios in a resulting synthesis gas.

It should be noted that the ratio of H₂ to CO in a synthesis gas can also be dependent on the water gas shift equilibrium. Although the stoichiometry in Equations (1)-(3) shows ratios of roughly 1 or roughly 3 for dry reforming and steam reforming, respectively, the equilibrium amounts of H₂ and CO in a synthesis gas can be different from the reaction stoichiometry. The equilibrium amounts can be determined based on the water gas shift equilibrium, which relates the concentrations of H₂, CO, CO₂ and H₂O based on the reaction shown in equation (4).

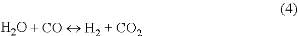

In some embodiments, the catalyst composition can also serve as water gas shift catalysts. Thus, if a reaction environment for producing H₂ and CO also includes H₂O and/or CO₂, the initial stoichiometry from the reforming reaction may be altered based on the water gas shift equilibrium. However, this equilibrium is also temperature dependent, with higher temperatures favoring production of CO and H₂O. As a result, the ratio of H₂ to CO that is generated when forming synthesis gas is constrained by the water gas shift equilibrium at the temperature in the reaction zone when the synthesis gas is produced.

The ability to adjust the H₂:CO molar ratio of the synthesis gas provides a flexible process that can be combined with a wide variety of synthesis gas upgrading processes. Illustrative synthesis gas upgrading processes can include, but are not limited to, Fischer-Tropsch processes, methanol and/or other alcohol synthesis, e.g., one or more C₁-C₄ alcohols, fermentation processes, separation processes that can separate hydrogen to produce a H₂-rich product, dimethyl ether, and combinations thereof. These synthesis gas upgrading processes are well-known to persons having ordinary skill in the art. In some embodiments, the upgraded product can include, but is not limited to, methanol, syncrude, diesel, lubricants, waxes, olefins, dimethyl ether, other chemicals, or any combination thereof.

Systems suitable for carrying out the reforming of the second hydrocarbon-containing feed can include systems that are well-known in the art such as the fixed bed reactors disclosed in WO Publication No. WO2017078894; the fluidized riser reactors and/or downer reactors disclosed in U.S. Pat. Nos. 3,888,762; 7,102,050; 7,195,741, 7,122,160; and 8,653,317, and U.S. Patent Application Publication Nos. 2004/0082824; 2008/0194891; and the reverse flow reactors disclosed in U.S. Pat. Nos. 7,740,829; 8,551,444; 8,754,276; 9,687,803; and 10,160,708; and U.S. Patent Application Publication Nos.: 2015/0065767 and 2017/0137285; and WO Publication No. WO2013169461.

### Feeds and Energy

The first and second hydrocarbon-containing feeds described herein can be derived either from fossil fuel or non-fossil fuel resources. For example, propane can be a product or by-product of a process using biomass as the feed. The fuels described in this work can also be derived either from fossil fuel or non-fossil fuel resources. For example, methane or H₂ can be a product or by-product of a process using biomass as the feed. The fuels described in this work can also be made from renewable energy such as renewable electricity. For example, renewable electricity can be used to produce H₂ through water electrolysis. The energy used in the processes described herein can also be provided by renewable electricity, instead of a fuel.

### EXAMPLES

The foregoing discussion can be further described with reference to the following non-limiting examples.

### Example I

An inventive catalyst composition (Ex. 1) was prepared according to the following procedure. An aqueous solution (200 ml) that contained 60 g of Mg(NO₃)₂·6H₂O (0.10 mol) and 30 g of Al(NO₃)₃·9H₂O (0.04 mol) was added dropwise to 200 mL of an aqueous solution that contained 10.6 g of Na₂CO₃ (0.05 mol) at room temperature while stirring. A 50% NaOH solution was added as needed to maintain a pH of 10 while the two solutions were combined (required 41.2 g of the 50% NaOH solution). The solution was then aged by stirring at room temperature for 16 hrs to produce a slurry or gel. After aging, the slurry or gel became thick and did not settle easily. The product was filtered and washed with water followed by ethanol, but not allowed to fully dry such that it remained a wet product. The wet product was added to 1000 mL of ethanol and stirred at room temperature for 4 hours. The product was filtered again, but not allowed to dry. The wet product was dried under vacuum at room temperature overnight, then at a temperature of 60° C. in air for 4 hours. The synthesis processes yielded 27.15 g of a fluffy white powder after room temperature vacuum that had a BET surface area of 365.7 m²/g. The fluffy white powder was calcined at 500° C. for 4 hrs in air to produce a plurality of mixed metal oxide support particles of Ex. 1. The mixed metal oxide support particles contained 70 wt % MgO and 30 wt % Al₂O₃ mixed on the atomic scale. The BET surface area of the mixed metal oxide support particles was 303.9 m²/g.

Tin (IV) chloride pentahydrate (0.103 g) (Acros Organics), chloroplatinic acid hexahydrate (0.0184 g) (BioXtra), and an appropriate amount of deionized water were mixed in a small glass vial to make a solution for incipient wetness impregnation. The plurality of mixed metal oxide support particles were impregnated with the solution. As such, the plurality of mixed Mg/Al metal oxide support particles had a surface area ≥300 m²/g when the Pt-containing compound and the promoter-containing compound were added thereto. The impregnated plurality of mixed metal oxide support particles were dried at a temperature of 110° C. for 6 hours in air and calcined at a temperature of 800° C. for 12 hours in air to produce the catalyst composition of Ex. 1. The catalyst composition of Ex. 1 contained nominally 0.3 wt % Pt and 1.5 wt % Sn.

A comparative catalyst composition (CEx.) 1 was prepared according to the following procedure. Tin (IV) chloride pentahydrate (0.103 g)(Acros Organics), chloroplatinic acid hexahydrate (0.0184 g) (BioXtra), and an appropriate amount of deionized water were mixed in a small glass vial to make a solution for incipient wetness impregnation. The solution was contacted with a plurality of mixed Mg/Al metal oxide support particles of CEx. 1 (2.3 g of PURALOX^{MG} 70/170 (Sasol), which was a MgO—Al₂O₃ mixed metal oxide that was produced by calcining hydrotalcite) to impregnate the plurality of support particles. The plurality of support particles was a mixed Mg/Al metal oxide that contained 70 wt % MgO and 30 wt % Al₂O₃. The BET surface area of the plurality of support particles was 170 m²/g according to Sasol. As such, the plurality of mixed Mg/Al metal oxide support particles had a surface area <300 m²/g when the Pt-containing compound and the promoter-containing compound were added thereto. The plurality of impregnated support particles was dried at 110° C. for 6 hours in air and calcined at 800° C. for 12 hours in air to produce the comparative catalyst composition of CEx, 1. The catalyst composition of CEx. 1 contained nominally 0.3 wt % Pt and 1.5 wt %/o Sn.

### Examples Using the Catalyst Compositions of Ex. 1 and CEx. 1

Fixed bed experiments that used the catalyst compositions of Ex. 1 and CEx. 1 were conducted at approximately 100 kPa-absolute. A gas chromatograph (GC)nwas used to measure the composition of the reactor effluents. The concentration of each component in the reactor effluents were used to calculate the C₃H₆ yield and selectivity. The C₃H₆ yield and selectivity, as reported in these examples, were calculated on the carbon mole basis.

In each example, 0.3 g of the catalyst composition was nixed with an appropriate amount of quartz diluent and loaded into a quartz reactor. The amount of diluent was determined so that the catalyst bed (catalyst+diluent) overlapped with the isothermal zone of the quartz reactor and the catalyst bed is largely isothermal during operation. The dead volume of the reactor was filled with quartz chips/rods.

The C₃H₆ yield and the selectivity at the beginning of tᵣₓₙ and at the end of tᵣₓₙ is denoted as Yᵢₙᵢ, Y_{end}, Sᵢₙᵢ, and S_{end}, respectively, and reported as percentages in the table below.

The process steps that used the catalyst compositions of Ex. 1 and CEx. 1 were as follows, 1. The system was flushed with an inert gas, 2. Dry air at a flow rate of 83.9 sccm was passed through a by-pass of the reaction zone, while an inert was passed through the reaction zone. The reaction zone was heated to a regeneration temperature of 800° C. 3. Dry air at a flow rate of 83.9 sccm was then passed through the reaction zone for 10 min to regenerate the catalyst. 4. The system was flushed with an inert gas. 5. A H₂ containing gas with 10 vol % H₂ and 90 vol % Ar at a flow rate of 46.6 sccm was passed through the by-pass of the reaction zone for a certain period of time, while an inert gas was passed through the reaction zone. This is then followed by flowing the H₂ containing gas through the reaction zone at 800° C. for 3 s. 6. The system was flushed with an inert gas. During this process, the temperature of the reaction zone was changed from 800° C., to a reaction temperature of 670° C. 7. A hydrocarbon-containing (HCgas) feed that included 81 vol % of C₃H₈, 9 vol % of inert gas (Ar or Kr) and 10 vol % of steam at a flow rate of 35.2 sccm was passed through the by-pass of the reaction zone for a certain period of time, while an inert gas was passed through the reaction zone. The hydrocarbon-containing feed was then passed through the reaction zone at 670° C. for 10 min. GC sampling of the reaction effluent started as soon as the feed was switched from the by-pass of the reaction zone to the reaction zone.

The above process steps were repeated in cycles until stable performance was obtained. Table1 shows that the catalyst composition of Ex. 1 exhibited a higher C₃H₆ yield than the catalyst composition of CEx. 1 at comparable selectivities during propane dehydrogenation.

TABLE 1

| | | Ex. 1 | CEx. 1 |
|---|---|---|---|
| Performance | Yᵢₙᵢ | 65.2 | 63.8 |
| | Y_{end} | 57.8 | 55.3 |
| | Sᵢₙᵢ | 96.0 | 95.9 |
| | S_{end} | 97.3 | 97.4 |

### Example 11

Additional inventive and comparative mixed Mg/Al metal oxide support particles were prepared according to the following procedures.

Inventive Support Ex. 2. An aqueous solution (50 ml) that contained 15.385 g Mg(NO₃)·6H₂O and 7.503 g Al(NO₃)₃·9H₂O was added dropwise to 50 mL aqueous solution that contained 2.65 g Na₂CO₃ at room temperature while stirring. A 50% NaOH solution was used to maintain a pH of 9.3 while the two solutions were combined (required 11.420 g of 50% NaOH solution). The solution was then aged by stirring at room temperature for 16 hrs to produce a slurry or gel. After aging, the slurry or gel was thick and did not settle easily. The product was then filtered and washed with water followed by ethanol, but not allowed to dry. The wet product was then added to 200 mL ethanol and stirred at room temperature for 4 hours. The product was filtered again, but not allowed to dry. The wet product was then dried under vacuum at room temperature overnight, then at 60° C. in air for 4 hours. The synthesis processes yielded 6.085 g of a fluffy white powder after room temperature vacuum. The fluffy white powder was calcined at 500° C. for 4 hrs in air to produce the plurality of mixed Mg/A metal oxide support particles of Ex. 2. The plurality of mixed Mg/Al metal oxide support particles contained 70 wt % MgO and 30 wt % Al₂O₃. The mixed Mg/Al metal oxide support particles had a surface area of 461.5 m²/g.

Inventive Support Ex. 3. A plurality of mixed Mg/Al metal oxide support particles were made similar to the Ex. 2 support particles except a 50% NaOH solution was used to maintain a pH of 8.5 while the two solutions were combined (required 10.207 g of 50% NaOH solution). The synthesis processes yielded 5.810 g of a fluffy white powder after room temperature vacuum that had a BET surface area of 510.2 m²/g. The fluffy white powder was calcined at a temperature of 350° C. for 4 hrs under vacuum to produce a plurality of mixed Mg/Al metal oxide support particles that contained 70 wt % MgO and 30 wt % Al₂O₃ and had a BET surface are of 441.2 m²/g.

Inventive Support Ex. 4. A plurality of mixed Mg/Al metal oxide support particles were made similar to the Ex. 2 support particles except a 50% NaOH solution was used to maintain a pH of 7.8 while the two solutions were combined (required 4.871 g of 50% NaOH solution). The synthesis processes yielded 3.132 g of a fluffy white powder after room temperature vacuum that had a BET surface area of 542.3 m²/g. The fluffy white powder was calcined at a temperature of 500° C. for 4 hrs in air to produce a plurality of mixed Mg/Al metal oxide support particles that contained 70 wt % MgO and 30 wt % Al₂O₃.

Inventive Support Ex 5. A plurality of mixed Mg/Al metal oxide support particles were made similar to the Ex. 2 support particles except a 50% NaOH solution was used to maintain a pH of 7.0 while the two solutions were combined (required 2.838 g of 50% NaOH solution). The synthesis processes yielded 2.344 g of a fluffy white powder after room temperature vacuum that had a BET surface area of 540.4 m²/g. The fluffy white powder was calcined at a temperature of 500° C. for 4 hrs in air to produce a plurality of mixed Mg/Al metal oxide support particles that contained 70 wt % MgO and 30 wt % Al₂O₃.

TABLE 2
Properties of Supports

| | pH When | Surf. | | |
|---|---|---|---|---|
| | Aqueous | Area | | Surf |
| | Solutions | After | Calcination | Area |
| | Were | Vacuum | Temp. | After |
| Example | Combined | (m²/g) | (° C.) | Calcination |
| Ex. 1 | 10 | 365.7 | 500 | 303.9 |
| Ex. 2 | 9.3 | 461.5 | 500 | 461.5 |
| Ex. 3 | 8.5 | 510.2 | 350 | 440.2 |
| Ex. 4 | 7.8 | 542.3 | 500 | n/a |
| Ex. 5 | 7 | 540.4 | 500 | n/a |

Comparative Support CEx. 2. An aqueous solution (100 ml) that contained 25.769 g Mg(NO₃)₂·6H₂O and 15.005 g Al(NO₃)3·9H₂O was added dropwise to 100 mL aqueous solution that contained 5.3 g Na₂CO₃ at room temperature while stirring. A 50% NaOH solution was used to maintain a pH of 10.3 while the two solutions were combined (required 21.660 g of 50% NaOH solution). The solution was then stirred at room temperature for 16 hrs. After aging, the slurry became a thick gel and did not settle easily. The product was then filtered and washed with 200 ml water without any organic solvent, e.g., ethanol. The product was then dried at 100° C. overnight to produce a plurality of dried support particles that had a BET surface area of 96 m²/g. The plurality of dried support particles were calcined at 500° C. for 4 hrs in air to obtain a plurality of mixed Mg/Al metal oxide support particles. The mixed Mg/Al metal oxide support particles contained 61 wt % MgO and 39 wt % Al₂O₃. The BET surface area of the mixed Mg/Al metal oxide support particles was 156.7 m²/g.

As can be seen from Table 2, when synthesis of the catalyst composition included contacting the slurry or gel with the organic solvent to displace at least a portion of the water therefrom to produce precursor support particles that included the organic solvent disposed thereon and/or therein and then removing at least a portion of the organic solvent therefrom to produce dried precursor support particles, such resulting catalyst composition had a surface area after calcination ≥300 m²/g, whereas when the organic solvent, e.g., ethanol, was not used (CEx. 2) the resulting catalyst composition had a surface area far less than 300 m²/g. i.e., 156.7 m²/g.

### Example III

Additional inventive mixed Mg/Al metal oxide support particles (Ex. 6, Ex. 7, and Ex. 8) were prepared according to the following procedure. An aqueous solution (100 ml) that contained 25.767 g Mg(NO₃)₂·6H₂O and 15.006 g Al(NO₃)₃·9H₂O was added dropwise to a 100 mL aqueous solution that contained 5.3 g Na₂CO₃ at room temperature while stirring. A 50% NaOH solution was used to maintain a pH of 8.5 while the two solutions were combined. The solution was then stirred at room temperature for 16 hrs. After aging, the slurry or gel was thick and did not settle easily. The product was filtered and washed with water followed by ethanol, but not allowed to dry. The wet product was then added to 400 mL ethanol and stirred at room temperature for 4 hours. The product was filtered again, but not allowed to dry. The wet product was then dried under vacuum at room temperature overnight, then at 60° C. in air for 4 hours to produce dried support particles of Ex. 6 that amounted to 10.412 g of a fluffy white pow der that had a surface area of 499.7 m²/g. The mixed metal oxide contained 76 wt % MgO and 24 wt % Al₂O₃. A first portion of the support particles of Ex. 6 were calcined at 500° C. for 4 hrs in air to obtain the support particles of Ex. 7 that had a surface area of 320.4 m²/g. A second portion of the support particles of Ex 6 were calcined at 650° C. for 4 hrs in air to obtain the support particles of Ex. 8 that had a surface area of 301.2 m²/g. As such, the Pt-containing compound and the promoter-containing compound could be independently contacted with any of the supports of Ex. 7, Ex. 8, and Ex. 9 to produce the catalyst composition disclosed herein.

### Example IV

Two additional supports (CEx. 3 and CEx. 4) were prepared according to the same synthesis procedure used to produce the support of Ex. 6. The only difference was that instead of drying under vacuum at room temperature, the wet product was dried in air at room temperature for 12 hours to produce the dried support particles of CEx. 3 and the wet product was dried in air at 100° C., 12 hours to produce the dried support particles of CEx. 4. The dried support particles of CEx. 3 and CEx. 4 had a surface area of only 262.2 m²/g and 257.2 m²/g, respectively, as compared to a surface area of 499.7 m²/g of the dried support particles of Ex. 6 that were produced by drying under a vacuum.

### Listing of Embodiments

This disclosure may further include the following non-limiting embodiments.

A1. A catalyst composition comprising up to 6 wt % of Pt and up to 10 wt % of a promoter comprising Sn, Cu, Au, Ag, Ga, a combination thereof, or a mixture thereof disposed on a support, wherein: the support comprises a mixed Mg/Al metal oxide, all weight percent values are based on the weight of the support, and when the Pt and the promoter are added to the support during synthesis of the catalyst composition, the support has a surface area ≥300 m²/g.

A2. The catalyst composition of paragraph A1, wherein, when the Pt and the promoter are added to the support, the support has a surface area ≥325 m²/g, ≥350 m²/g, ≥375 m²/g, 400 m²/g, ≥425 m²/g, or ≥450 m²/g.

A3. The catalyst composition of paragraph Al or paragraph A2, wherein the promoter comprises Sn.

A4. The catalyst composition of any of paragraphs A1 to A3, further comprising an alkali metal element comprising Li, Na, K, Rb. Cs, or a combination thereof, or a mixture thereof disposed on the support in an amount of up to 5 wt % based on the weight of the support.

A5. The catalyst composition of any of paragraphs A1 to A4, wherein a weight ratio of the Mg to the Al in the mixed Mg/Al metal oxide is in a range from 0.001, 0.01, 0.1, 1, 2, or 3 to 4, 5, 6, 12.5, 100, or 1,000.

A6. The catalyst composition of any one of paragraphs A1 to A5, wherein: the promoter comprises Sn, the support has a surface area ≥303 m²/g, and a weight ratio of Mg to Al in the mixed metal oxide is in a range from 2 to 6.

A7. The catalyst composition of any one of paragraphs A1 to A6, wherein the catalyst composition is in the form of particles having a size and a particle density that is consistent with a Geldart A or Geldart B definition of a fluidizable solid.

B1. A process for upgrading a hydrocarbon, comprising: (1) contacting a hydrocarbon-containing feed with a catalyst composition comprising Pt and a promoter disposed on a support to effect one or more of dehydrogenation, dehydroaromatization, and dehydrocyclization of at least a portion of the hydrocarbon-containing feed to produce a coked catalyst composition and an effluent comprising one or more upgraded hydrocarbons and molecular hydrogen, wherein: the hydrocarbon-containing feed comprises one or more of C₂-C₁₆ linear or branched alkanes, or one or more of C₄-C₁₆ cyclic alkanes, or one or more C₈-C₁₆ alkyl aromatics, or a mixture thereof, the catalyst composition comprises up to 6 wt % of Pt and up to 10 wt % of the promoter, based on the weight of the support, the promoter comprises Sn, Cu, Au, Ag. Ga, a combination thereof, or a mixture thereof, the support comprises a mixed Mg/Al metal oxide, and when the Pt and the promoter are added to the support during synthesis of the catalyst composition, the support has a surface area ≥300 m²/g.

B2. The process of paragraph B1, wherein the hydrocarbon-containing feed and the catalyst composition are contacted at a temperature in a range of from 300° C., to 900° C. under a hydrocarbon partial pressure of at least 20 kPa-absolute, wherein the hydrocarbon partial pressure is the total partial pressure of any C₂-C₁₆ alkanes and any C₈-C₁₆ alkyl aromatics in the hydrocarbon-containing feed.

B3. The process of paragraph B1 or paragraph B2, further comprising: (11) contacting at least a portion of the coked catalyst composition with an oxidant to effect combustion of at least a portion of the coke to produce a regenerated catalyst composition lean in coke and a combustion gas, and (III) contacting an additional quantity of the hydrocarbon-containing feed with at least a portion of the regenerated catalyst composition to produce a re-coked catalyst composition and additional effluent.

CL A process for upgrading a hydrocarbon, comprising: (1) contacting a hydrocarbon-containing feed with a catalyst composition comprising Pt and a promoter disposed on a support to effect reforming of at least a portion of the hydrocarbon-containing feed to produce a coked catalyst composition and a synthesis gas comprising H₂ and CO, wherein: the hydrocarbon-containing feed comprises one or more C₁-C₁₆ hydrocarbons and H₂O, CO₂, or a mixture of H₂O and CO₂, the hydrocarbon-containing feed and catalyst composition are contacted at a temperature of 400° C., or more, the catalyst composition comprises up to 6 wt % of Pt and up to 10 wt % of the promoter, based on the weight of the support, the promoter comprises Sn, Cu, Au, Ag, Ga, a combination thereof, or a mixture thereof, the support comprises a mixed Mg/Al metal oxide, and the support has a surface area >300 m²/g.

C2. A process for upgrading a hydrocarbon, comprising: (1) contacting a hydrocarbon-containing feed with a catalyst composition comprising Pt and a promoter disposed on a support to effect reforming of at least a portion of the hydrocarbon-containing feed to produce a coked catalyst composition and a synthesis gas comprising H₂ and CO, wherein: the hydrocarbon-containing feed comprises one or more C₁-C₁₀ hydrocarbons and H₂O, CO₂, or a mixture of H₂O and CO₂, the hydrocarbon-containing feed and catalyst composition are contacted at a temperature of 400° C., or more, the catalyst composition comprises up to 6 wt % of Pt and up to 10 wt % of the promoter, based on the weight of the support, the promoter comprises Sn. Cu, Au, Ag, Ga, a combination thereof, or a mixture thereof, the support comprises a mixed Mg/Al metal oxide, and when the Pt and the promoter are added to the support during synthesis of the catalyst composition, the support has a surface area ≥300 m²/g.

C3. The process of paragraph C1 or paragraph C2, further comprising (II) contacting at least a portion of the coked catalyst composition with an oxidant to effect combustion of at least a portion of the coke to produce a regenerated catalyst composition lean in coke and a combustion gas.

C4. The process of paragraph C3, further comprising (Ill) contacting a fuel with the oxidant and the coked catalyst composition to effect combustion of at least a portion of the fuel.

C5. The process of paragraph C3 or paragraph C4, further comprising (IV) contacting an additional quantity of the hydrocarbon-containing feed with at least a portion of the regenerated catalyst composition to produce a re-coked catalyst composition and additional effluent.

C6 The process of any one of paragraphs C1 to C5, wherein the hydrocarbon-containing feed is contacted with the catalyst composition in a fluidized bed reactor.

C7. The process of any one of paragraphs C1 to C5, wherein the hydrocarbon-containing feed is contacted with the catalyst composition in a fixed bed reactor.

C8. The process of any one of paragraphs C1 to C5, wherein the hydrocarbon-containing feed is contacted with the catalyst composition in a reverse flow reactor.

C9. The process of any one of paragraphs C1 to C8, wherein the promoter comprises Sn.

C10. The process of any one of paragraphs C1 to C9, wherein a weight ratio of the Mg to the Al in the mixed Mg/Al metal oxide is in a range from 0.001, 0.01, 0.1, 1.2, or 3 to 4, 5, 6, 12.5, 100, or 1,000.

C11. The process of any one of paragraphs C1 to C9, wherein the catalyst composition further comprises an alkali metal element comprising Li, Na, K, Rb, Cs, a combination thereof, or a mixture thereof disposed on the support in an amount of up to 5 wt % based on the weight of the support.

C12. The process of any one of paragraphs C1 to C11, further comprising at least one of: reacting at least a portion of the synthesis gas under effective Fischer-Tropsch conditions in the presence of a Fischer-Tropsch catalyst to produce an upgraded product, wherein the Fischer-Tropsch catalyst comprises a shifting Fischer-Tropsch catalyst or a non-shifting Fischer-Tropsch catalyst, subjecting at least a portion of the synthesis gas to a fermentation process to produce an alcohol, an organic acid, or a mixture thereof; contacting at least a portion of the synthesis gas with a catalyst to produce at least one C₁-C₄ alcohol; and separating H₂ from the synthesis gas to produce a H₂-rich product.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A catalyst composition comprising a support and, based on the weight of the support, up to 6 wt % of Pt and up to 10 wt % of a promoter comprising Sn, Cu, Au, Ag, Ga, a combination thereof, or a mixture thereof disposed on the support, wherein:
the support comprises a mixed Mg/Al metal oxide, and
the support has a surface area >300 m²/g.

2. The catalyst composition of claim 1, wherein the support has a surface area ≥350 m²/g.

3. The catalyst composition of claim 1, wherein the promoter comprises Sn.

4. The catalyst composition of claim 1, further comprising an alkali metal element comprising Li, Na, K, Rb, Cs, or a combination thereof, or a mixture thereof disposed on the support in an amount of up to 5 wt % based on the weight of the support.

5. The catalyst composition of claim 1, wherein a weight ratio of the Mg to the Al in the mixed Mg/Al metal oxide is in a range from 0.001 to 1,000.

6. The catalyst composition of claim 1, wherein:
the promoter comprises Sn,
the support has a surface area ≥303 m²/g, and
a weight ratio of Mg to Al in the mixed metal oxide is in a range from 2 to 6.

7. The catalyst composition of claim 1, wherein the catalyst composition is in the form of particles having a size and a particle density that is consistent with a Geldart A or Geldart B definition of a fluidizable solid.

8. A process for making a catalyst composition, comprising:
(I) combining a first compound comprising Mg, a second compound comprising Al, a first base compound, water, and optionally a Pt-containing compound and/or a promoter-containing compound under agitation conditions for a time sufficient to produce a slurry or a gel comprising a plurality of precursor support particles comprising water disposed thereon and/or therein;
(II) contacting the slurry or the gel with an organic solvent to displace at least a portion of the water from the slurry or the gel to produce a plurality of precursor support particles comprising the organic solvent disposed thereon and/or therein, wherein optionally a Pt-containing compound and/or a promoter-containing compound is contacted with (i) the slurry or the gel or (ii) with the plurality of precursor support particles comprising the organic solvent disposed thereon and/or therein;
(III) removing at least a portion of the organic solvent from the plurality of precursor support particles to produce a plurality of dried precursor support particles, wherein optionally a Pt-containing compound and/or a promoter-containing compound is contacted with the dried precursor support particles; and
(IV) heating the plurality of dried precursor support particles to a temperature of at least 350° C., to produce a plurality of calcined support particles, wherein optionally a Pt-containing compound and/or a promoter-containing compound is contacted with the plurality of calcined support particles, wherein:
the Pt-containing compound and the promoter-containing compound are independently present in at least one of steps (I), (II), (III), and (IV),
the catalyst composition comprises up to 6 wt % of Pt and up to 10 wt % of the promoter, based on the weight of the plurality of calcined support particles,
the promoter comprises Sn, Cu, Au, Ag, Ga, a combination thereof, or a mixture thereof,
the plurality of calcined support particles comprises a mixed Mg/Al metal oxide, and
the plurality of calcined support particles has a surface area ≥300 m²/g.

9. The process of claim 8, wherein the Pt-containing compound and the promoter-containing compound are contacted with the plurality of calcined support particles.

10. The process of claim 8, wherein step (I) comprises:
(Ia) preparing a first aqueous solution comprising the first compound and the second compound;
(Ib) preparing a second aqueous solution comprising the first base compound, wherein the second aqueous solution has a pH≥7; and
(Ic) combining the first and second aqueous solutions under the agitation conditions for the time sufficient to produce the slurry or the gel.

11. The process of claim 8, wherein a pH during step (I) is maintained greater than 7 by adding a second base compound thereto.

12. The process of claim 11, wherein the first base compound and the second base compound are the same or different with respect to one another.

13. The process of claim 11, wherein the first base compound comprises sodium carbonate, sodium nitrate, sodium chloride, sodium sulfate, or a mixture thereof, and wherein the second base compound comprises sodium hydroxide, ammonium hydroxide, potassium hydroxide, urea, a hexamethyl tetraamine, or a mixture thereof.

14. The process of claim 8, wherein step (I) is carried out at a temperature ≤150° C.

15. The process of claim 8, wherein step (II) comprises:
(IIa) filtering and washing the slurry or the gel with a first quantity of the organic solvent to produce a first filtered product; and
(IIb) combining the first filtered product with a second quantity of the organic solvent under agitation conditions for a time period in a range from 30 minutes to 12 hours to produce the plurality of precursor support particles comprising the organic solvent disposed thereon and/or therein.

16. The process of claim 15, wherein a composition of the first quantity of the organic solvent and a composition of the second quantity of the organic solvent is the same or different with respect to one another, and wherein the first and second quantities of organic solvent independently comprise ethanol, propanol, butanol, pentanol, acetone, ethyl acetate, or a mixture thereof.

17. The process of claim 15, wherein a weight ratio of the second quantity of organic solvent to the first filtered product is in a range from 0.1:1 to 30:1.

18. The process of claim 8, wherein step (III) comprises drying the plurality of precursor support particles (i) under an atmosphere comprising nitrogen, argon, carbon dioxide, methane, air, or a mixture thereof or (ii) under a vacuum in the substantial absence of an atmosphere at a temperature in a range from 20° C., to 340° C. for a time period in a range from 1 hour to 24 hours to produce the plurality of dried precursor support particles.

19. The process of claim 8, wherein step (III) comprises:
(IIIa) drying the plurality of precursor support particles under a vacuum at a temperature in a range from 20° C., to 340° C. for a time period in a range from 1 hour to 24 hours to produce a plurality of pre-dried precursor support particles, and
(IIIb) drying the plurality of pre-dried precursor support particles under a pressure of at least atmospheric pressure at a temperature in a range from 20° C., to 340° C. for a time period in a range from 1 hour to 24 hours to produce the plurality of dried precursor support particles.

20. The process of claim 19, wherein the plurality of pre-dried precursor support particles is dried in step (IIIb) under an atmosphere comprising, oxygen, nitrogen, argon, carbon dioxide, methane, air, or a mixture thereof.

21. The process of claim 8, wherein step (IV) comprises contacting the plurality of calcined support particles with at least one of the Pt-containing compound and the promoter-containing compound to produce a plurality of Pt-containing and/or promoter-containing calcined support particles, the process further comprising (V) calcining the plurality of the Pt-containing and/or the promoter-containing calcined support particles to produce re-calcined support particles having the Pt and the promoter disposed thereon, wherein the catalyst composition comprises the re-calcined support particles.

22. The process of claim 8, further comprising optionally combining an alkali metal element comprising Li, Na, K, Rb, Cs, a compound thereof, a combination thereof, or a mixture thereof in step (I), (II), (III), and/or (IV) such that the catalyst composition further comprises up to 5 wt % of the alkali metal element disposed on the calcined support particles, based on the weight of the plurality of calcined support particles.

23. A process for upgrading a hydrocarbon, comprising:
(I) contacting a hydrocarbon-containing feed with a catalyst composition comprising Pt and a promoter disposed on a support to effect one or more of dehydrogenation, dehydroaromatization, and dehydrocyclization of at least a portion of the hydrocarbon-containing feed to produce a coked catalyst composition and an effluent comprising one or more upgraded hydrocarbons and molecular hydrogen, wherein:
the hydrocarbon-containing feed comprises one or more of C2-C16 linear or branched alkanes, or one or more of C4-C16 cyclic alkanes, or one or more C8-C16 alkyl aromatics, or a mixture thereof,
the catalyst composition comprises up to 6 wt % of Pt and up to 10 wt % of the promoter, based on the weight of the support,
the promoter comprises Sn, Cu, Au, Ag, Ga, a combination thereof, or a mixture thereof,
the support comprises a mixed Mg/Al metal oxide, and
the support has a surface area ≥300 m²/g.

24. The process of claim 23, wherein the hydrocarbon-containing feed and the catalyst composition are contacted at a temperature in a range of from 300° C., to 900° C. under a hydrocarbon partial pressure of at least 20 kPa-absolute, wherein the hydrocarbon partial pressure is the total partial pressure of any C2-C16 alkanes and any C8-C16 alkyl aromatics in the hydrocarbon-containing feed.

25. The process of claim 23, further comprising:
(II) contacting at least a portion of the coked catalyst composition with an oxidant to effect combustion of at least a portion of the coke to produce a regenerated catalyst composition lean in coke and a combustion gas; and
(III) contacting an additional quantity of the hydrocarbon-containing feed with at least a portion of the regenerated catalyst composition to produce a re-coked catalyst composition and additional effluent.
